(19) United States
(12) Patent Application Publication
(10) Pub. No.: US 2007/0009441 A1
(43) Pub. Date: Jan. 11, 2007

(54) Biodegradable nanoparticles

(75) Inventors: Nandanan Erathodiyil, Singapore (SG); G. Ramachandra Reddy, Novi, MI (US); Young Wan Ham, Novi, MI (US)

Correspondence Address: STERNE, KESSLER, GOLDSTEIN & FOX PLLC, 1100 NEW YORK AVENUE, N.W., WASHINGTON, DC, 20005, US

(73) Assignee: Molecular Therapeutics, Inc., Ann Arbor, MI (US)

(21) Appl. No.: 11/176,595

(22) Filed: Jul. 8, 2005

Related U.S. Application Data
(60) Provisional application No. 60/585,889, filed on Jul. 8, 2004.

(51) Int. Cl. A61K 49/10; A61K 9/14

(52) U.S. Cl. 424/9.34; 424/489; 977/906

(57) ABSTRACT

The present invention relates to polymeric nanoparticles useful in drug and agent delivery, as well as for imaging, diagnosis and targeting. The polymeric nanoparticles of the present invention comprise polymers and cross-linkers that, when degraded, leave simple nontoxic biocompatible molecules that can be metabolized, excreted, or absorbed by the body. The present invention also relates to processes for producing the polymeric nanoparticles of the present invention, and methods of using them in drug and agent delivery, as well as imaging, diagnosis and targeting.

### CROSS REFERENCE TO RELATED APPLICATIONS

The present application claims the benefit of the filing date of U.S. Provisional Patent Application No. 60/585,889, filed Jul. 8, 2004, the disclosure of which application is incorporated by reference herein in its entirety.

### BACKGROUND OF THE INVENTION

1. Field of the Invention

The present invention relates to polymeric nanoparticles useful in drug and agent delivery, as well as for imaging, diagnosis and targeting. The polymeric nanoparticles of the present invention comprise polymers and cross-linkers that, when degraded, leave simple nontoxic biocompatible molecules that can be metabolized, excreted, or absorbed by the body. The present invention also relates to processes for producing the polymeric nanoparticles of the present invention, and methods of using them in drug and agent delivery, as well as imaging, diagnosis and targeting.

2. Related Art

Due to their small size, polymeric nanoparticles have been found to evade recognition and uptake by the reticulo-endothelial system (RES), and thus can circulate in the blood for an extended period. (Borchard, G. et al., Pharm Res. 7:1055-1058 (1996)). In addition, nanoparticles are able to extravasate at the pathological site, such as the leaky vasculature of a solid tumor, providing a passive targeting mechanism. (Yuan F. et al., Cancer Research 55:3752-3756 (1995); Duncan, R. et al., STP Pharma. Sci. 4:237 (1996).) U.S. Pat. No. 6,322,817 to Maitra et al., discloses the production of nanoparticles comprised of polymeric micelles containing the anticancer drug paclitaxel. The '817 patent describes the use of amphiphilic monomers in conjunction with a cross-linking agent to create the encapsulating micelles. The cross-linking agents disclosed in the '817 patent however, are not biodegradable.

U.S. Pat. No. 6,143,558 to Kopelman et al., describes polymeric nanoparticles for use as optical probes for monitoring the response of cells to various external stimuli and insults. The nanoparticles of the '558 patent are not biodegradable and retain their contents, thereby allowing external monitoring of cellular responses.

U.S. Pat. No. 6,528,575 to Schade et al., relates to the use of cross-linked copolymers obtainable by precipitation polymerization of monomer mixtures comprising (a) monoethylenically unsaturated C₃-C₈ carboxylic acids, their anhydrides or mixtures of carboxylic acids and anhydrides, (b) compounds with at least 2 non-conjugated ethylenic double bonds in the molecule as cross-linkers and, where appropriate, (c) other monoethylenically unsaturated monomers which are copolymerizable with monomers (a) and (b).

An important feature of any nanoparticle, especially for agent delivery, is the biocompatibility of the particle. This requires that the polymer particle degrade after some period so that it can be excreted, metabolized or absorbed by the body. These criteria require polymer compositions that are well tolerated. In addition, controlled polymer degradation also allows for increased levels of agent delivery to a diseased site.

However, to date there remain few degradable nanoparticles composed of well-tolerated polymers. The present invention fulfills this need by providing polymers and cross-linked polymeric nanoparticles that degrade into simple nontoxic molecules that can be easily metabolized, absorbed or excreted from the body. The nanoparticles of the present invention can be used for patient diagnosis and imaging as well as in various treatments in therapies, and the degradable nature of the nanoparticles allow them to deliver enhanced amounts of encapsulated contents at the disease site.

### BRIEF SUMMARY OF THE INVENTION

In one embodiment, the present invention provides processes for producing polymeric nanoparticles comprising condensing one or more primary dihydroxy compounds and one or more diacids to generate a polyester; adding one or more cross-linkers selected from the group consisting of ethylene glycol diitaconate, glycerol (bis) itaconate, sorbitol diitaconate, glycerol dimethacrylate and divinyl citrate; initiating polymerization to generate a solid particle; and removing the solid particle from solution.

In suitable embodiments, the condensation occurs via esterification, for example via enzyme catalysis, including lipase catalysis. The processes of the present invention suitably further comprise passing the solid particle through one or more porous filters to generate a nanoparticle that is less than 200 nm in diameter. In certain embodiments, initiation of polymerization occurs in the presence of one or more surfactants.

In other embodiments, the processes of the present invention further comprise adding an agent to be encapsulated to the solution prior to initiation of polymerization and/or adding a functionalized monomer to generate a functionalized group on the surface of the nanoparticle.

In certain embodiments, the primary dihydroxy compound used in the practice of the present invention is selected from the group consisting of sorbitol, mannitol, iditol, sucrose, fructose, maltose, ribose, lactose, glycerol, ethylene glycol, propylene glycol and glycerol. In suitable embodiments, the diacid is selected from the group consisting of itaconic acid, adipic acid, succinic acid, fumaric acid and acylamidoglutamic acid.

The present invention also provides processes for producing polymeric monomers via esterification of one or more hydroxyacid compounds, such as gluconic acid; hydroxy aliphatic acids, such as glycolic acid, lactic acid and acrylamidoglycolic acid; hydroxy aromatic acids; salicylic acid; glyceric acid; threonic acid; serine; and glutathione. Such polymeric monomers can be utilized in the various methods disclosed herein to produce polymeric nanoparticles.

The present invention also provides polymeric nanoparticles produced by the processes of the present invention. Suitably, these nanoparticles are biodegradable.

In other embodiments, the polymeric nanoparticles of the present invention further comprise a functionalized surface group, e.g. an amine group, a thiol group, an alcohol group or a carboxylic acid group, and this functionalized surface group can be bound to a targeting ligand, suitably an antibody or peptide.

In suitable embodiments, the polymeric nanoparticles of the present invention encapsulate one or more water-soluble agents, including a small organic molecule drug, a DNA molecule, an RNA molecule, a protein, a fluorescent dye, a radioisotope, a contrast agent, and an imaging agent. In other embodiments, the polymeric nanoparticles encapsulate one or more water-insoluble agents. Suitably, the nanoparticles of the present invention encapsulate paclitaxel, gemcitabine, a gadolinium complex or a gadolinium chelate or iron oxide. In certain suitable embodiments, the polymeric nanoparticles of the present invention are less than 200 nm in diameter.

In an embodiment, the present invention provides polymeric nanoparticles made by the processes of the present invention, wherein sorbitol and itaconate are condensed to form the polymeric monomers and the cross-linker is ethylene glycol diitaconate. The present invention also provides polymeric nanoparticles made by the processes of the present invention, wherein gluconic acid and acrylamidoglycolic acid are condensed to form the polymeric monomers and the cross-linker is glycerol dimethacrylate.

In another embodiment, the present invention provides processes for producing polymeric nanoparticles comprising condensing one or more primary hydroxyacid compounds to generate a polyester; adding one or more cross-linkers selected from the group consisting of glycerol dimethacrylate, ethylene glycol diitaconate, glycerol (bis) itaconate, sorbitol diitaconate and divinyl citrate; initiating polymerization to generate a solid particle; and removing the solid particle from solution.

The present invention also provides processes for producing polymeric nanoparticles comprising: condensing one or more primary dihydroxy compounds and one or more diacids to generate a polyester; or condensing one or more primary hydroxyacid compounds to generate a polyester; adding one or more water-soluble cross-linkers; initiating polymerization to generate a solid particle; and removing the solid particle from solution. Suitable water-soluble cross-linkers for use in the practice of the present invention include, but are not limited to, lysine-diacrylamide, diethylenetriamine-diacrylamide, arginine-diacrylamide and 2,2′-oxydiethanol-diacrylate.

The present invention also provides methods of treating a tumor in a mammalian patient comprising: administering to the patient a polymeric nanoparticle of the present invention, wherein the polymeric nanoparticle encapsulates one or more cancer chemotherapeutic agents. In a related embodiment, the present invention provides methods of treating a tumor in a mammalian patient comprising: administering to the patient a polymeric nanoparticle of the present invention; and administering ionizing radiation to the patient, wherein the polymeric nanoparticle encapsulates one or more radiation-sensitizing agents. The present invention also provides methods of imaging the polymeric nanoparticles in a mammalian patient. In addition, the present invention provides pharmaceutical compositions comprising one or more nanoparticles and one or more pharmaceutically acceptable excipients.

### BRIEF DESCRIPTION OF THE DRAWINGS

FIG. 1 shows intensity weighted Gaussian particle size distribution of sorbitol-itaconate polymeric nanoparticles produced in accordance with one embodiment of the present invention.

FIG. 2 shows intensity weighted NICOMP particle size distribution of sorbitol-itaconate polymeric nanoparticles produced in accordance with one embodiment of the present invention.

FIG. 3 shows a synthesis scheme for production of poly gluconic-acrylamidoglycolate nanoparticles in accordance with one embodiment of the present invention.

FIG. 4 shows a synthesis scheme for production of poly Sorbitol-glycerol dimethacrylate nanoparticles in accordance with one embodiment of the present invention.

FIG. 5 shows the degradation profile of Ru-encapsulated sorbitol itaconate nanoparticles in 1N NaOH over a period of 36 hours.

FIG. 6 shows the degradation of sorbitol-itaconate nanoparticles in PBS by particle sizing over a period of 15 days.

FIG. 7 shows the relative concentration timecourse in normal brain, tumor and vessel, and brain/tumor signal-to-noise ratio after i.v. bolus injection of iron oxide (FeOX) nanoparticles (uptake).

FIG. 8 shows the extended relative concentration time course in normal brain, tumor, and vessel, and brain/tumor signal-to-noise ratio after i.v. bolus injection of FeOX nanoparticles (clearance).

FIGS. 9a-9f show (a) MRI anatomical scout image of a rat brain, (b) MRI image pre-injection, (c) MRI image post-injection at 10 minutes, (d) MRI image post-injection at 80 minutes, (e) MRI image post injection at 2 hours and (f) MRI image post-injection at 36 hours.

FIG. 10 shows the degradation of FITC conjugated sorbitol-itaconate nanoparticles in PBS at 37° C. over a period of 12 days.

FIG. 11 shows the synthesis of FITC-Sorbitol nanoparticle conjugates according to one embodiment of the present invention.

FIG. 12 shows the conjugation of sulfo-SMCC to fluorescent labeled nanoparticles according to one embodiment of the present invention.

FIG. 13 shows the synthesis of nanoparticles and the F3-peptide-2-iminothiolane conjugate according to one embodiment of the present invention.

FIG. 14 shows the synthesis of FITC-SMCC conjugated nanoparticles with the F3-peptide-2-IT conjugate according to one embodiment of the present invention.

FIG. 15 shows the synthesis of FITC-Fe₃O₄-Sorbitol nanoparticle conjugates according to one embodiment of the present invention.

FIG. 16 shows the conjugation of sulfo-SMCC to fluorescent labeled Fe3O4-nanoparticles according to one embodiment of the present invention.

FIG. 17 shows the synthesis of FITC-SMCC conjugated, Fe3O4 encapsulating, nanoparticles with the F3-peptide-2-IT conjugate according to one embodiment of the present invention.

FIG. 18 shows a synthesis scheme for preparing poly sorbitol-itaconic acid nanoparticles linked with a water-soluble, lysine-diacrylamide cross-linker.

### DETAILED DESCRIPTION OF THE INVENTION

Suitable embodiments of the present invention are now described. While specific configurations and arrangements are discussed, it should be understood that this is done for illustrative purposes only. A person skilled in the relevant art will recognize that other configurations and arrangements can be used without departing from the spirit and scope of the invention.

The present invention provides polymeric nanoparticles (referred to interchangeably herein as “nanoparticle(s)”) comprising a backbone polymer and a polymeric cross-linker that links two or more of the backbone polymers. Suitably, the nanoparticles of the present invention are used for drug and agent delivery, as well as for disease diagnosis and medical imaging in human and animal patients. The nanoparticles of the present invention can also be used in invasive and non-invasive therapies, such as radiation therapy, boron-neutron capture therapy and magnetic based therapies. The nanoparticles of the present invention can also be used in other applications such as chemical or biological reactions where a reservoir or depot is required.

As used herein, the term “nanoparticle” refers to particles between about 10 nm and about 1000 nm in diameter. In suitable embodiments, the diameter of the nanoparticles of the present invention will be less than about 200 nm in diameter, and more suitably less than about 100 nm in diameter. In certain such embodiments, the nanoparticles of the present invention will be between about 10 nm and about 200 nm, between about 30 nm and about 100 nm, or between about 40 nm and about 80 nm in diameter. As used herein, when referring to any numerical value, “about” means a value of ±10% of the stated value (e.g. “about 100 nm” encompasses a range of diameters from 90 nm to 110 nm, inclusive).

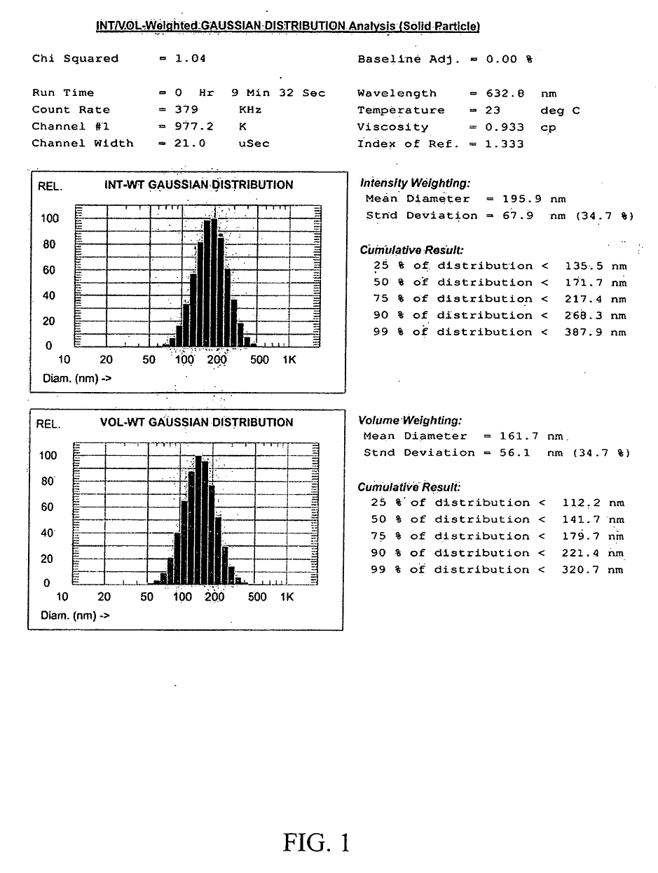

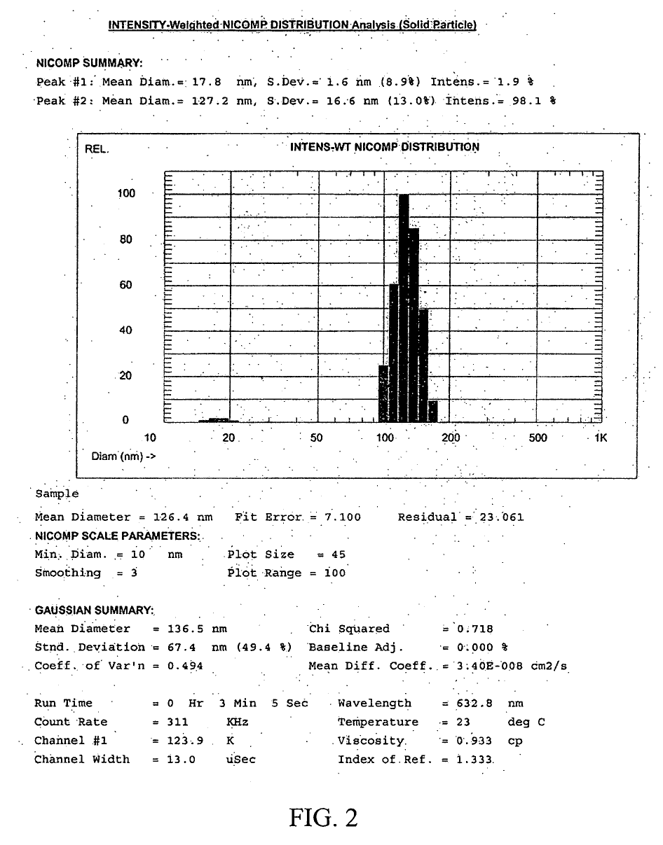

FIGS. 1 and 2 represent particle size of the nanoparticles of the present invention demonstrating their fairly uniform size distribution and diameter. The small size of the nanoparticles of the present invention will allow them to evade capture by the RES, as well as extravasate from the vasculature, specifically in diseased areas such as the leaky vasculature of solid tumors.

In one embodiment, the present invention provides processes for producing polymeric nanoparticles comprising condensing one or more primary dihydroxy compounds and one or more diacids to generate one or more polymeric monomers, adding one or more cross-linkers, initiating polymerization to generate a solid particle, the particle comprising a polymeric backbone of the polymeric monomers cross-linked with the polymeric cross-linkers and removing the solid particle from solution.

In another embodiment, the present invention provides processes for producing nanoparticles comprising condensation of one or more primary hydroxyacid compounds to generate one or more degradable polymeric monomers and initiating polymerization in the presence of one or more cross-linkers to generate solid nanoparticles.

In another embodiment, the present invention provides polymeric nanoparticles made by any of the processes of the present invention.

The terms “backbone” or “backbone polymer” as used herein refer to the polymer units that make up the linear structure of the primary polymer component of the nanoparticles. Suitable backbone polymers for use in the practice of the present invention include but are not limited to, polyesters made by lipase catalysis and derived from natural sugars or diols such as, but not limited to, sorbitol, mannitol, iditol, cyclic sugars (such as sucrose, fructose, lactose, ribose and maltose), glycerol, ethylene glycol, propylene glycol, glycerol, etc. The lipase catalyzed synthesis of various polyesters has been described in Macromolecules 36:9804 (2003); Macromolecules 36:8219 (2003); and Biomacromolecules 34:544 (2003), the disclosures of which are incorporated herein by reference in their entireties. Suitable diacids useful in the practice of the present invention include, but are not limited to, adipic acid, itaconic acid, sebacic acid, succinic acid, maleic acid, tartaric acid, fumaric acid, itaconic acid, lactic acid, glutamic acid, etc. In suitable embodiments of the present invention, polysorbitol itaconate is used as a backbone polymer. In other embodiments, the backbone is polysorbitol itaconate containing one or more diacids (sebacic acid, adipic acid, etc.). Additional backbone polymers include monomers produced by esterification of one or more hydroxyacid compounds, such as gluconic acid; hydroxy aliphatic acids, such as glycolic acid, lactic acid and acrylamidoglycolic acid; hydroxy aromatic acids; salicylic acid; glyceric acid; threonic acid; serine; and glutathione. In other embodiments of the present invention, the polymeric backbones can comprise polyamides, for example, produced via the reaction of lysine or serine and a diacid compound.

The nanoparticles of the present invention also comprise a cross-linker that forms links between two or more of the backbone polymers. Suitable polymeric cross-linkers for use in the practice of the present invention include, but are not limited to, glycerol diitaconate, sorbitol diitaconate, ethylene glycol diitaconate, glycerol (bis) acrylate (GBA), glycerol (bis) itaconate, 3-(acryloyloxy)-2-hydroxypropyl methacrylate, ethylene glycol diacrylate, glycerol dimethacrylate, and divinyl citrate. Additional cross-linkers include water-soluble cross linkers, such as those known the art. Exemplary water-soluble, cross-linkers include, but are not limited to lysine-diacrylamide, diethylenetriamine-diacrylamide, arginine-diacrylamide and 2,2′-oxydiethanol-diacrylate.

The skilled artisan will readily recognize that the addition of other charged groups onto the various water-soluble cross-linkers disclosed will increase the water solubility of these cross-linkers, and such variations are encompassed by the present invention.

In suitable embodiments of the present invention when the backbone polymer is polysorbitol itaconate, the polymeric cross-linker is ethyleneglycol diitaconate. Another embodiment is represented below showing a polysorbitol itaconate backbone cross-linked with sorbitol diitaconate (structure shown is for illustrative purposes only and may not represent the exact structure of the polymers).

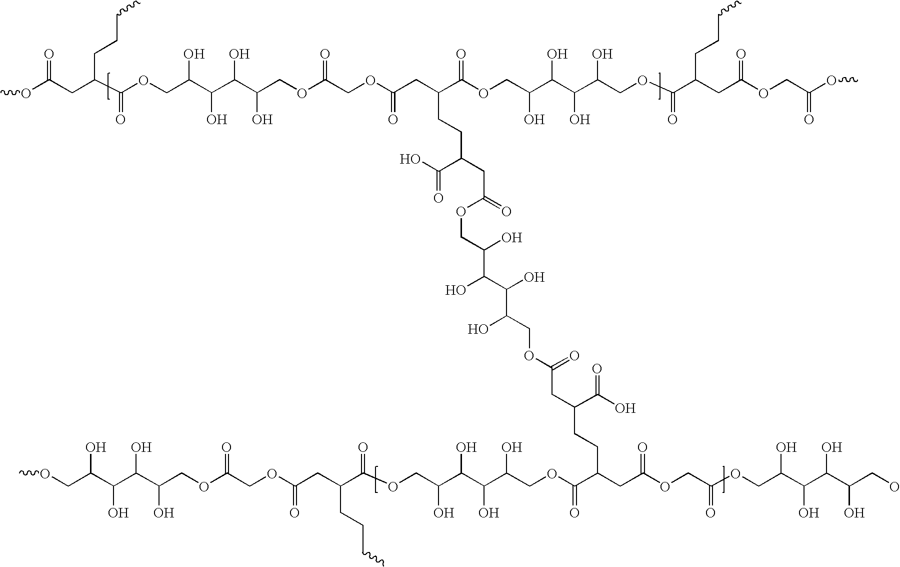

The polymeric nanoparticles of the present invention are prepared so as to be degradable, and suitably, to be biodegradable. The term “biodegradable” as used herein refers to both enzymatic and non-enzymatic breakdown or degradation of the polymeric structure. The back-bone polymers and/or the polymeric cross-linkers utilized in the present invention provide specific degradation points where breakdown of the polymeric cross-linker can occur. Suitably, these degradation points will be carboxylic acid ester groups, though other biodegradable groups can be used in accordance with the present invention as can be determined by the ordinarily skilled artisan. When the nanoparticles of the present invention come in contact with the proteins, enzymes and hydrolyzing chemicals found in blood and other biological fluids, the back-bone polymers and/or the polymeric cross-linkers are broken down. This degradation creates linear polymeric end products that can be readily excreted from the body, metabolized, or otherwise absorbed by the body. The degradation also provides for a method via which encapsulated contents, such as drugs or other agents, can be released at a site within the body. By selecting the proper back-bone polymer and/or polymeric cross-linker with a desired rate of degradation, the rate of release of encapsulated contents from the nanoparticles can be controlled. Varying the amount of cross-linker (e.g. 5%, 10%, 15%, 20%, 25%, or 30%) relative to backbone monomer will also allow for tailoring of the release rate of the encapsulated agent.

In other suitable embodiments of the present invention, the nanoparticles comprise functionalized surface groups. Certain such functionalized surface groups include, but are not limited to, amine groups, hydroxyl groups, thiol groups, alcohol groups, carboxylic acid groups and other acidic groups. Such functional groups allow the addition of targeting molecules to the surface of the nanoparticles for enhanced site-specific delivery of the nanoparticles. Such targeting molecules include, but are not limited to antibody molecules, growth receptor ligands (e.g., EGF, FGF, PDGF, VEGF, erb-B2), asialoglycoprotein and other targeting molecules known to those skilled in the art, such as targeting peptides and polypeptides. Drug molecules can also be attached to the functionalized molecules on the surface of the nanoparticles.

In related embodiments, the nanoparticles of the present invention further comprise polymeric coatings on their surface that create a stearic barrier to the approach of biological proteins, thereby prolonging blood circulation time. Such polymer coatings include poly(ethylene glycol) (PEG), suitably 500-5000 molecular weight, grafted to the surface. In certain such embodiments, these PEG molecules further comprise targeting molecules attached to their ends that facilitate delivery and targeting of the nanoparticles.

In suitable embodiments of the present invention, the nanoparticles comprise one or more water-soluble, or water-insoluble agents, encapsulated inside. In addition, a water-soluble or water-insoluble agent can be attached to the surface nanoparticle via methods well known in the art.

Suitable water-soluble and water-insoluble agents that can be encapsulated within the interior of the nanoparticles include small organic molecule drugs such as chemotherapeutic agents and their prodrugs, including, but not limited to, alkylating agents such as busulfan, cis-platin, mitomycin C, and carboplatin; antimitotic agents such as colchicine, vinblastine, paclitaxel (e.g., TAXOL®), and docetaxel; topoisomerase I inhibitors such as camptothecin and topotecan; topoisomerase II inhibitors such as doxorubicin and etoposide; RNA/DNA antimetabolites such as 5-azacytidine, 5-fluorouracil and methotrexate; DNA antimetabolites such as 5-fluoro-2′-deoxy-uridine, ara-C, hydroxyurea, gemcitabine, capecitabine and thioguanine; antibodies such as HERCEPTIN® and RITUXAN®, as well as other known chemotherapeutics such as photofrin, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine. Additional water-soluble and water-insoluble drugs can also be encapsulated in the nanoparticles of the present invention.

The nanoparticles of the present invention can also be used to encapsulate DNA, RNA, and other proteins and polymers. Suitable such proteins and polymers will be less than about 10 nm in size.

In certain embodiments of the present invention, the nanoparticles can be used to encapsulate one or more fluorescent dyes, such as carboxyfluorescein, ruthenium, and rhodamine; one or more radioisotopes; one or more Magnetic Resonance Imaging (MRI) contrast agents, such as iron oxide (e.g., superparamagnetic iron oxide (SPIO)); or one or more contrast agents. For example, Gadolinium (Gd) complexes or Gadolinium chelates (e.g., Gadolinium DTPA, Gd DOTA, Gadomer-17) and the polymers of such materials can be incorporated. Such contrast agents can be either chemically attached to the surface of the nanoparticles or encapsulated. Similarly, polyiodinated compounds can be incorporated in the nanoparticles. In addition, ¹⁰B enriched compounds can be incorporated in the degradable nanoparticles for BNCT studies. These agents can be used to allow for identification the nanoparticles in vivo in human and animal patients. Gadolinium-complexes can also be used in for Neutron Capture Therapy (Gd-NCT) applications. See e.g., Matsumura, A., et al., Anticancer Res. 23:2451-2456 (2003), Shikata F., et al., Eur. J. Pharm. Biopharm. 53:57-63 (2002) and Tokumitsu H., et al., Cancer Lett. 150:177-182 (2000).

In other suitable embodiments, the nanoparticles of the present invention comprise two or more different agents from the groups described throughout. For example, the nanoparticles of the present invention can incorporate a combination of agents including, a chemotherapeutic agent, a radioisotope, and an imaging or contrast agent encapsulated within the same nanoparticle. Suitably, this nanoparticle can then be surface modified to incorporate a PEG coating and/or an antibody or other targeting molecule on its surface.

In suitable embodiments of the processes of the present invention, the monomers used to generate the polymeric backbone comprise a primary dihydroxy compound, including, but not limited to, polyesters made by lipase catalysis and derived from natural sugars or diols such as sorbitol, mannitol, iditol, cyclic sugars (such as sucrose, fructose, lactose, ribose and maltose), glycerol, ethylene glycol, propylene glycol, glycerol, etc., and a diacid. Suitable diacids include, but are not limited to, adipic acid, itaconic acid, sebacic acid, succinic acid, maleic acid, tartaric acid, fumaric acid, itaconic acid, lactic acid, glutamic acid, etc. Polymeric cross-linkers useful in the practice of the processes of the present invention include, but are not limited to, glycerol diitaconate, sorbitol diitaconate, ethylene glycol diitaconate, glycerol (bis) acrylate (GBA), glycerol (bis) itaconate, 3-(acryloyloxy)-2-hydroxypropyl methacrylate, ethylene glycol diacrylate, glycerol dimethacrylate, and divinyl citrate. Additional backbone polymers include monomers produced by esterification of one or more hydroxyacid compounds, such as gluconic acid; hydroxy-alkyl-carboxylic acids; hydroxy aliphatic acids, such as glycolic acid, lactic acid and acrylamidoglycolic acid; hydroxy aromatic acids; salicylic acid; glyceric acid; threonic acid; serine; and glutathione. Water-soluble cross linkers can also be used in the practice of the present invention. Suitable water-soluble cross-linkers include those known in the art, including, but not limited to lysine-diacrylamide, diethylenetriamine-diacrylamide, arginine-diacrylamide and 2,2′-oxydiethanol-diacrylate.

In certain embodiments of the present invention, a water-based solution is formed of polymeric monomers and cross-linkers, suitably a sodium phosphate buffer, though non-water based solutions can also be used. Polymerization can be initiated by any initiation protocol known to those skilled in the art. Condensation of the dihydroxy and diacid molecules (or condensation of the hydroxyacid compounds) to generate the polyester that makes up the polymeric backbone monomers of certain embodiments suitably occurs via esterification. In certain suitable embodiments this condensation can occur via enzyme catalysis (e.g. lipase catalysis). For example, NOVOZYM®-435 beads can be used as catalysts. In other embodiments, ammonium persulfate and N,N,N′,N′-tetramethylethylenediamine (TEMED) are used to initiate polymerization and generate the cross-linked polymers. Polymerization generates cross-links between the polymeric backbone comprised of monomer units, and the cross-linking molecules, to generate a cross-linked polymer network.

In certain embodiments, polymerization is carried out in the presence of surface active agents and suitable solvents (such as hexane) under microemulsion conditions. Suitable surface active agents include lipids and surfactants (including anionic, cationic, zwitterionic and non-ionic surfactants). Exemplary surfactants include, but are not limited to, dioctyl sulfosuccinate (AOT or Aerosol OT), Brij 30, and the like. Suitable concentrations of surface active agents and solvents (as well as backbone polymers and cross-linkers) can be readily determined by the ordinarily skilled artisan. In certain embodiments, two surfactants, e.g. both AOT and Brij 30, can be used together at varying ratios, e.g. 0.001 to 1, 0.01 to 1, 0.1 to 1, 0.5 to 1, 1 to 1, 2 to 1, 5 to 1, 10 to 1, etc, AOT to Brij 30. The ordinarily skilled artisan will readily recognize that the amount and composition of the surfactants and solvents used can be varied according to reaction conditions and components.

Following polymerization, the solid particles that are formed are filtered and washed, and then dried. The solid particles can then be suspended in a water-based solution, and filtered or extruded through one or more filters with an appropriate pore size, to generate nanoparticles that are less than about 200 nm in diameter.

In suitable embodiments of the present invention, a water-soluble, or water-insoluble agent can be added to the solution of polymer monomer units and cross-linkers prior to initiation of polymerization. Following polymerization, a solid particle is generated that has the agent encapsulated within its interior. Suitable agents for encapsulation are described throughout the present specification and well known by those skilled in the art.

In certain embodiments, the processes of the present invention further comprise the generation of a functional group on the surface of the nanoparticle. Suitably this functional group can be an amine group or carboxylic acid group of another monomer that can be added to the solution prior to polymerization. In other suitable embodiments, the processes of the present invention further comprise the addition of a PEG or antibody molecule to the surface of the nanoparticle.

The following represent a few non-limiting examples of combinations of polymeric monomers and cross-linkers that can be used to create nanoparticles according to the processes of present invention:

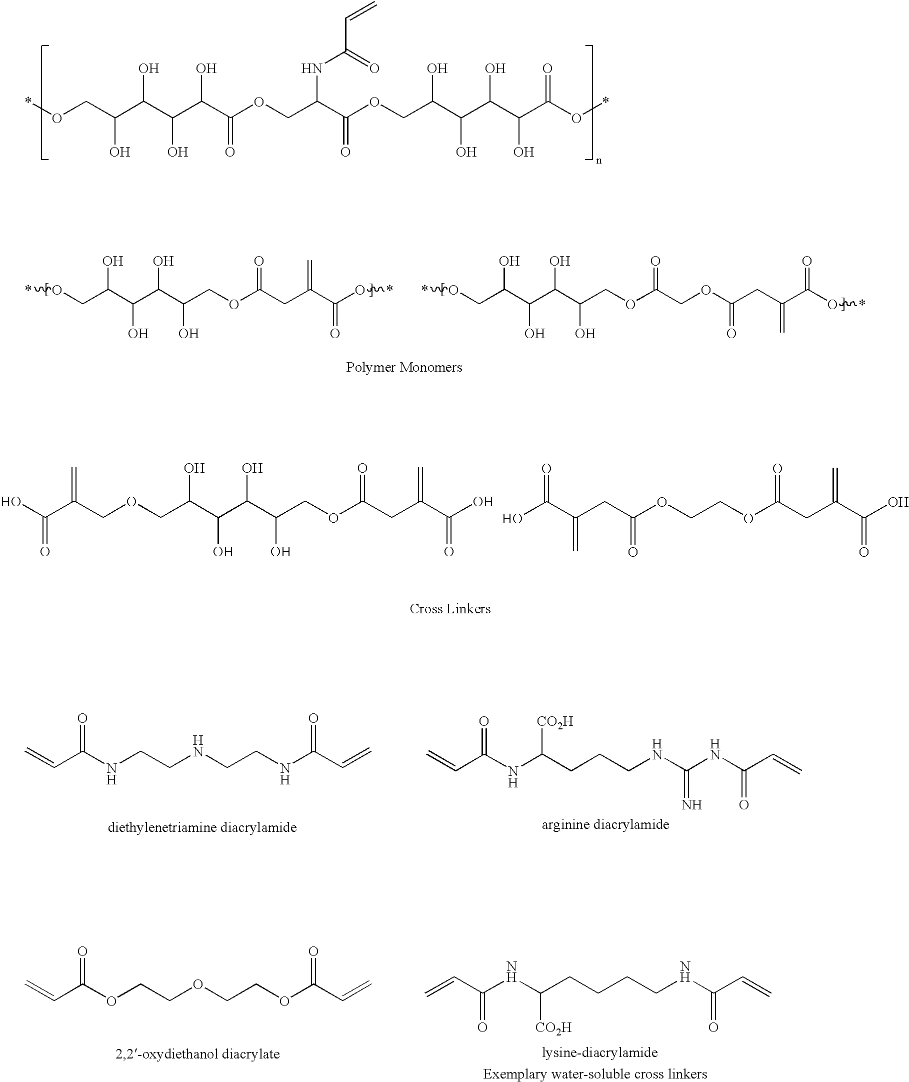

The nanoparticles of the present invention can suitably be used for delivery of agents to a diseased site in the body of an animal, particularly a mammal, including a human, and in the diagnosis or imaging of a specific tissue or site in the animal's body. In suitable embodiments, the nanoparticles can encapsulate several agents, including chemotherapeutic agents, contrast agents, and radioisotopes, within the same nanoparticle. These nanoparticles can further comprise targeting molecules on their surface. The nanoparticles of the present invention are especially useful for the treatment, diagnosis and imaging of solid tumors, including, but not limited to, cancers of the brain, breast, limbs, lung, heart, and gut. The nanoparticles of the present invention can also be used in the treatment, diagnosis and imaging of cardiovascular and infectious diseases, as well other medical conditions where such nanoparticles would be useful.

The polymeric nanoparticles can be used for various methods of treatment and/or diagnosis in human and animal patients. In certain embodiments, the present invention provides methods of treating a tumor in a mammalian patient comprising: administering to the patient a polymeric nanoparticle according to the present invention, wherein the polymeric nanoparticle encapsulates one or more cancer chemotherapeutic agents. Suitable chemotherapeutic agents include those known in the art and disclosed throughout, and include gemcitabine and photofrin. In suitable embodiments, the nanoparticle can further encapsulate an imaging agent. Imaging agents that can be encapsulated are well known in the art and include those disclosed throughout, such as iron oxide. The present invention also provides methods of imaging the polymeric nanoparticles which encapsulate imaging agents.

In other embodiments, the polymeric nanoparticles can be used to treat tumors by encapsulating a photodynamic therapeutic drug within the targeted nanoparticle. In certain embodiments, the present investigation provides methods to encapsulate photofrin, a photodynamic therapeutic agent, in a targeted nanoparticle and evaluating the efficacy of the therapy by diffusion MRI.

In other embodiments, the polymeric nanoparticles can be used to deliver radiation-sensitizing agents to tumors. In such embodiments, polymeric nanoparticles encapsulating one or more radiation-sensitizing agents are administered to a patient in need of such treatment and ionizing radiation is administered to the patient. Suitably, the radiation-sensitizing agents are released from the nanoparticles at the tumor site such that the ionizing radiation can act upon the agents at the tumor site. Radiation-sensitizing agents include any agent that increases the sensitivity of a tumor to ionizing radiation and include, but are not limited to, gemcitabine, paclitaxel, carboplatin, and other such compounds. In other embodiments, an imaging agent, such as those described herein, can be co-encapsulated with the radiation-sensitizing agent (or attached to the surface of the nanoparticle) to allow for imaging of the nanoparticles prior to and/or during radiation treatment. The nanoparticles can also comprise a targeting molecule, such as those described herein, to allow for targeting of the nanoparticles to the tumor tissue.

The present invention also provides pharmaceutical compositions comprising the nanoparticles of the present invention. The compositions may include a physiologically or pharmaceutically acceptable carrier, excipient, or stabilizer in addition to the nanoparticles. The term “pharmaceutically acceptable” means a non-toxic material that does not interfere with the effectiveness of the biological activity of the active agents. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active agent is combined to facilitate the application. The components of the pharmaceutical compositions also are capable of being commingled with the compounds of the present invention, and with each other, in a manner such that there is no interaction which would substantially impair the desired pharmaceutical efficiency. Suitable excipients include, but are not limited to, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP).

It will be readily apparent to one of ordinary skill in the relevant arts that other suitable modifications and adaptations to the methods and applications described herein can be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLE 1

Synthesis of Sorbitol-Itaconic Acid Polyester

Sorbitol (1.82 g, 10 mmol) and itaconic acid (1.3 g, 10 mmol) were transferred into a 100 mL round bottom flask. The reactants were heated with stirring to 140° C. and the mixture melted. The temperature of the reaction mixture was then lowered to 90-95° C. and the reaction components remained as a viscous liquid. Then, NOVOZYM®-435 beads (Novozymes, Denmark) (10% wt/wt relative to monomers, 310 mg, dried at 25° C./10 mmHg/24 hrs) were added. Within 2 hrs the reaction mixture appeared monophasic with suspended catalyst beads. The flask was sealed with a rubber septum and the reaction was maintained at 90° C. with mixing. After the first 6 hrs of the reaction, the contents of the reaction were maintained under reduced pressure (40 mmHg). The polymerization was terminated after 48 h by dissolving the reaction mixture in methanol, removing the enzyme by filtration, and stripping off the solvent in vacuo. The product was then dried in a vacuum oven (10 mmHg, 30° C., 24 h).

### EXAMPLE 2

Synthesis of Gluconic-Acrylamidoglycolate (Gluconic-AGA) Polyester

A mixture of D-gluconic acid (11.7 g, 0.06 mol) and acrylamidoglycolic acid (3.26 g, 0.02 mol) in a 100 mL round bottom flask was heated to 160° C. to obtain a melt. The temperature of the reaction mixture was lowered to 90-95° C. and then NOVOZYM®-435 beads (Novozymes, Denmark) (10% wt/wt relative to monomers, (1.5 g), dried at 25° C./10 mmHg/24 hrs) were added. The temperature of the reaction mixture was maintained at 90° C. with occasional mixing. After 6 hrs the reaction mixture was subjected to vacuum (40 mmHg) while maintaining the temperature at 90° C. for 48 h. The reaction mixture was cooled to room temperature, the polymer was extracted into methanol, and the beads were removed by filtration. The filtrate was concentrated under reduced pressure and the product (thick liquid) was further subjected to a high vacuum (24 h) to give 9.9 g of pale yellow liquid.

### EXAMPLE 3

Synthesis of Poly Sorbitol-Itaconic Acid Nanoparticles

A clean 20 ml glass vial was charged with sorbitol itaconate polymer (1.5 g) and 4 ml of sodium phosphate buffer (10 mM, pH 7.3). The suspension was sonicated for 2 min to obtain a clear solution. Ethylene glycol diitaconate (0.5 g, 25 wt % of the polymeric monomer) was added to the reaction mixture and sonicated for an additional 5 min. The resulting slightly turbid monomer solution was added to a 250 ml round bottom flask containing an argon-purged, well stirred solution of dioctyl sulfosuccinate (AOT or Aerosol AT) (3.2 g) and Brij 30 (6.4 ml) in hexanes (100 ml). After a 10 min stirring under an argon blanket at room temperature, the reaction mixture was treated with freshly prepared aqueous ammonium persulfate (65 μl, 10%) and N,N,N′,N′-tetramethylethylenediamine (TEMED) (85 μl) to initiate polymerization. The reaction mixture was gently stirred at room temperature overnight to ensure complete polymerization.

The reaction mixture was then concentrated to a thick residue and re-suspended in ethanol (100 ml). The precipitated particles were filtered and thoroughly washed with ethanol (5×160 ml) in an Amicon stirred cell equipped with a Biomax filter membrane (500 Kda, filtration pressure 10 psi, nitrogen). The solid material was transferred onto a Whatman filter paper, gently crushed into a fine powder, and subjected to air-drying until a constant weight was observed (3-4 hrs). (Typical yield around 100%.) The product (white free-flowing powder) can be stored at 4° C. for extended periods of time.

The product was suspended in water (20 mg/mL) and sonicated to get a homogenous solution. The solution was transferred into an Amicon stirred cell equipped with a Biomax (500 KDa) filter membrane and thoroughly washed with water (5×150 ml). The concentrated sample (˜50 mg/ml) was passed through 0.45 μm and then 0.2 μm filters and stored at 4° C. until further use.

### EXAMPLE 4

Synthesis of Gluconic-Acrylamidoglycolate Nanoparticles

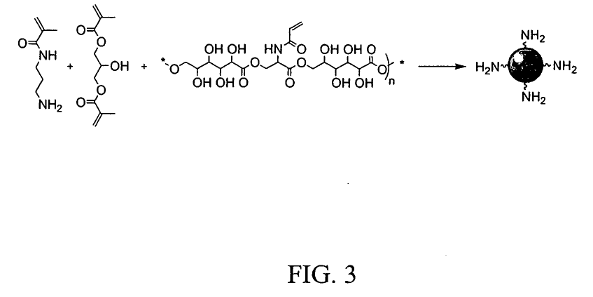

A representative synthesis scheme is shown in FIG. 3. A clean 20 ml glass vial was charged with gluconic-AGA polymer (2.3 g) and 4 ml of sodium phosphate buffer (10 mM, pH 7.3). The suspension was sonicated for 2 min to obtain a clear solution. Glycerol dimethacrylate (0.4 g, 0.0017 mol) and 3-aminopropyl methacrylamide (0.2 g, 0.001 mol) were added to the polymer solution and the mixture was sonicated for an additional 5 min. The resulting slightly turbid monomer solution was added to a 250 ml round bottom flask containing an argon-purged, well stirred solution of dioctyl sulfosuccinate (AOT) (3.6 g) and Brij 30 (3.8 ml) in hexanes (100 ml). After a 10 min stirring under an argon blanket, the polymerization was initiated by adding freshly prepared aqueous ammonium persulfate (65 μl, 10%) and N,N,N′,N′-tetramethylethylenediamine (TEMED) (85 μl). The reaction mixture was gently stirred at room temperature overnight to ensure complete polymerization.

The reaction mixture was then concentrated to a thick residue and re-suspended in ethanol (100 ml). The precipitated particles were filtered and thoroughly washed with ethanol (5×180 ml) in an Amicon stirred cell equipped with a Biomax filter membrane (500 Kda, filtration pressure 10 psi, nitrogen). The solid material was transferred onto a Whatman filter paper, gently crushed into a fine powder, and subjected to air-drying until a constant weight was observed (3-4 hrs). The yield of the product was 2.7 g (white powder).

### EXAMPLE 5

Synthesis of poly Sorbitol-Glycerol dimethacrylate Nanoparticles

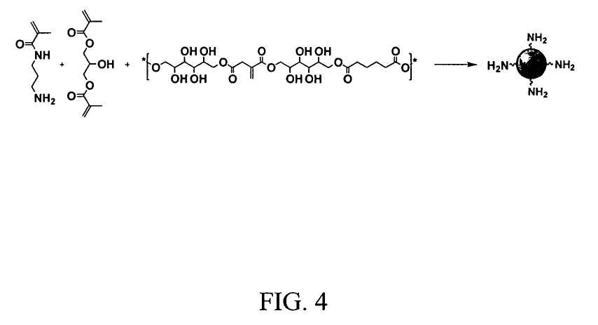

A representative reaction scheme is shown in FIG. 4. A clean 20 ml glass vial was charged with Sorbitol-Adipic acid-Itaconic acid (SAI 1:0.4:0.6) polymer (2.0 g) and 4 ml of sodium phosphate buffer (10 mM, pH 7.3). The suspension was sonicated for 2 min to obtain a clear solution. Glycerol dimethacrylate (0.36 g, 0.0016 mol) and 3-aminopropyl methacrylamide (0.2 g, 0.001 mol) were added to the polymer solution and the mixture was sonicated for an additional 5 min. The resulting slightly turbid monomer solution was added to a 250 ml round bottom flask containing an argon-purged, well stirred solution of dioctyl sulfosuccinate (AOT) (3.6 g) and Brij 30 (3.8 ml) in hexanes (100 ml). After a 10 min stirring under an argon blanket, the polymerization was initiated by adding freshly prepared aqueous ammonium persulfate (65 μl, 10%) and N,N,N′,N′-tetramethylethylenediamine (TEMED) (85 μl). The reaction mixture was gently stirred at room temperature overnight to ensure complete polymerization.

The reaction mixture was then concentrated to a thick residue and re-suspended in ethanol (100 ml). The precipitated particles were filtered and thoroughly washed with ethanol (5×180 ml) in an Amicon stirred cell equipped with a Biomax filter membrane (500 Kda, filtration pressure 10 psi, nitrogen). The solid material was transferred onto a Whatman filter paper, gently crushed into a fine powder, and subjected to air-drying until a constant weight was observed (3-4 hrs). The yield of the product was 1.66 g (white powder).

### EXAMPLE 6

Synthesis of Amine Functionalized Degradable Sorbitol Itaconate Nanoparticles

The monomer solution was prepared by adding sorbitol itaconate polymer (2.5 g), N-(3-aminopropyl)-methacrylamide (0.5 g) and ethylene glycol diitaconate (1.0 g) to sodium phosphate buffer (8 ml, 10 mM, pH 7.3). The slightly turbid mixture was sonicated for 10 min and added to a solution containing AOT (6.4 g) and Brij 30 (12.8 ml) in argon purged hexanes (180 mL). After a 10 min stirring at ambient temperature, the polymerization reaction was initiated by treating with a freshly prepared aqueous ammonium persulfate (130 μl, 10%) and TEMED (170 μl). The reaction mixture was stirred overnight under an argon atmosphere.

Hexane was removed under reduced pressure and the residue was treated with ethanol (150 ml). The precipitated nanoparticle solution was transferred into an amicon stirred cell (200 ml) equipped with a 500 KDa Biomax filter membrane (filtration pressure 10 psi, nitrogen), washed thoroughly with ethanol (5×160 ml) and air dried. The solid material was gently crushed to a fine free-flowing white powder (yield 100%). The product was storable at 4° C. for extended periods of time.

The product was suspended in water (20 mg/ml) and sonicated to give a homogenous solution. The solution was transferred into an amicon stirred cell equipped with a Biomax (500 KDa) filter membrane and thoroughly washed with water (5×150 ml). The concentrated sample (˜50 mg/ml) was passed through 0.45 μm and then 0.2 μm filters and stored at 4° C. until further use.

### EXAMPLE 7

Synthesis of Nanoparticles Encapsulating Iron Oxide

A 20 ml glass vial was charged with sorbitol itaconate polymer (1.5 g) and 2 ml of sodium phosphate buffer (10 mM, pH 7.3). The suspension was sonicated for 2 min to obtain a clear solution. Ethylene glycol diitaconate (0.5 g) was added to the reaction mixture and sonicated for an additional 5 min. The resulting slightly turbid monomer solution was treated with iron oxide solution (2 ml, EMG 805, Ferrotec) and the deep dark mixture was sonicated for 10 min.

A 250 ml round bottom flask equipped with a mechanical stirrer was charged with AOT (3.2 g) and Brij 30 (6.4 ml) in argon purged hexanes (100 ml). The clear solution was treated with the above iron oxide monomer solution with stirring. After a 10 min mechanical stirring (high speed) under an argon blanket at room temperature, the polymerization was initiated by treating the reaction mixture with freshly prepared aqueous ammonium persulfate (65 μl, 10%) and N,N,N′,N′-tetramethylethylenediamine (TEMED) (85 μl). The reaction mixture was stirred at room temperature overnight.

The solvent was removed under reduced pressure to obtain a black thick residue. The resulting thick residue was re-suspended in ethanol (100 mL) and the precipitated nanoparticles were filtered and thoroughly washed with ethanol (5×160 ml) in an Amicon stirred cell (200 ml) equipped with a Biomax filter membrane (500 Kda, filtration pressure 10 psi, nitrogen). The solid material was transferred onto a Whatman filter paper, gently crushed into a fine powder and subjected to air-drying until a constant weight was observed (3-4 hrs). The product (Black free-flowing powder) was storable at 4° C. for extended periods of time.

The product was suspended in water (20 mg/ml) and sonicated to give a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45 μm and 0.2 μm filters and stored at 4° C. until further use.

### EXAMPLE 8

Synthesis of Photofrin (or Ruthenium dye) Encapsulated Nanoparticles

A clean 20 ml glass vial was charged with sorbitol itaconate polymer (1.5 g) and 4 ml of sodium phosphate buffer (10 mM, pH 7.3). The suspension was sonicated for 2 min to obtain a clear solution. Ethylene glycol diitaconate (0.5 g) was added to the reaction mixture and sonicated for an additional 5 min. Photofrin (or Ruthenium dye (Ru)) was added and the mixture was sonicated for an additional 5 min. The resulting slightly turbid monomer solution was added to a 250 ml round bottom flask containing an argon-purged, well stirred solution of dioctyl sulfosuccinate (3.2 g) and Brij 30 (6.4 ml) in hexanes (100 ml). After a 10 min stirring under an argon blanket at room temperature, the reaction mixture was treated with freshly prepared aqueous ammonium persulfate (65 μl, 10%) and N,N,N′,N′-tetramethylethylenediamine (TEMED) (85 μl) to initiate the polymerization. The reaction mixture was gently stirred at room temperature overnight to ensure complete polymerization.

The reaction mixture was concentrated to a thick residue and re-suspended in ethanol (100 ml). The precipitated particles were filtered and thoroughly washed with ethanol (5×160 ml) in an Amicon stirred cell equipped with a Biomax filter membrane (500 Kda, filtration pressure 10 psi, nitrogen). The solid material was transferred onto a Whatman filter paper, gently crushed into a fine powder and subjected to air-drying until a constant weight was observed (3-4 hrs). The product (dark brown free-flowing powder in case of photofrin and light pink powder for Ru dye) was storable at 4° C. for extended periods of time.

The product was suspended in water (20 mg/ml) and sonicated to get a homogenous solution. The solution was transferred into an amicon stirred cell equipped with a Biomax (500 Kda) filter membrane and thoroughly washed with water (5×150 ml). The concentrated sample (˜50 mg/ml) was passed through 0.45 μm and 0.2 μm filters and stored at 4° C. until further use.

### EXAMPLE 9

Degradation of Sorbitol-itaconate Nanoparticles

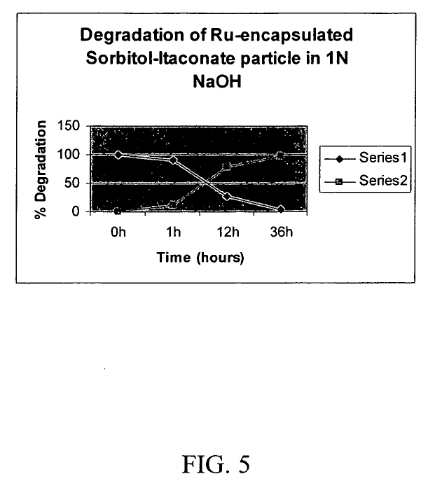

100 mg of Ru-loaded sorbitol itaconate nanoparticles were suspended in 10 ml PBS and filtered. The residue was treated with 10 mL of 1 M sodium hydroxide incubated for 12 h intervals and filtered. FIG. 5, Series 1 shows the decrease in Ru-dye content in the particle over time and Series 2 shows the increase in Ru-dye content in the filtrate over time.

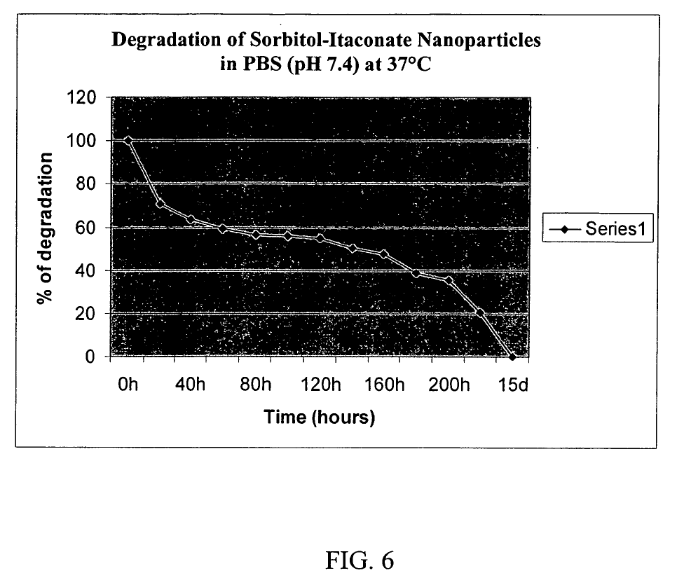

100 mg of blank sorbitol itaconate nanoparticles were suspended in 10 ml PBS (pH 7.3) and filtered. The residue was incubated at 37° C. and the particles sizes were measured at regular intervals. FIG. 6 shows the degradation profile of sorbitol itaconate particles in PBS over a period of 15 days.

### EXAMPLE 10

Cytotoxicity of Sorbitol Itaconate Nanoparticles

MTT Assay and Toxicity

The MTT assay was used for the quantitation of in vitro tumor cell chemosensitivity, for the assessment of photoradiation therapy, and for the screening of anticancer compounds. The assay is based on the cleavage of the yellow 3-(4,5-Dimethylthiazol-2-yl)-2,5diphenyltetrazolium bromide (MTT) into purple formazan by metabolically active cells. The MTT formazan crystals were insoluble in aqueous solution, but were solubilized by adding the solubilization solution consisting of 50% DMF and 50% SDS (20% pH 4.7), then incubating the plates overnight in a humidified atmosphere (e.g., 37° C., 5% CO₂). The solubilized formazan product was photometrically quantitated using an ELISA reader. An increase in the number of living cells results in an increase of total metabolic activity which leads to a stronger color formation, thus, a lower absorbance reading on the ELISA reader for a given well indicated a lower number of living cells in that well.

In vitro cytotoxicity was determined using the MTT assay. 9L cells were plated in triplicate at a density of 100,000 cells/ml of DMEM containing 10% fetal bovine serum in 96-well plates. Nanoparticle preparation with drug was filtered in DMEM without FBS and then passed through 0.22 μm syringe filter. Two-fold dilutions of nanoparticles in DMEM were added to 9L cells in a 96 well plate. After 48 hrs of incubation, 100 μl of MTT solution (2.5 mg/ml in PBS) was added to each well, and the plates were incubated for 1-2 h at 37° C. and then solubilized with 100 μl of a solution containing 50% of DMF/20% SDS pH 4.7 was added. The live cell number was quantified by measuring light absorbance (490 nm) in a automated microplate reader. The absorbance values of control untreated cells were compared with the absorbance of cells treated with different concentration of nanoparticles containing drugs. IC₅₀ was determined by treating cells with different concentrations of drug. All particles were found to be nontoxic in vitro.

### EXAMPLE 11

Magnetic Resonance Imaging of Fe₃O₄ Encapsulated Sorbitol Itaconate Nanoparticles

Animal Model

Intracerebral 9L tumors were induced in male Fischer 344 rats weighing between 125 and 150 g. Briefly, 9L cells (10⁵) were implanted in the right forebrain at a depth of 3 mm through a 1 mm burr hole. The surgical field was cleaned with 70% ethanol and the burr hole was filled with bone wax to prevent extracerebral extension of the tumor. Animals were imaged using Magnetic Resonance Imaging (MRI) beginning at 14 days post cell implantation to select tumors between 60 and 80 μl in volume for in vivo NP studies.

Nanoparticle Administration

Nanoparticles were administered to rats as a suspension (1.5 ml; 100 mg/ml) in normal saline by tail vein injection at a dose of 150/0.25 mg nanoparticles/kg body weight. An Angiocath™ Teflon catheter was placed in the tail vein of the animal and flushed with 10 units/ml of heparin and a pre-primed infusion line was connected to the ANGIOCATH™. The nanoparticles were injected over 45 seconds during dynamic MR scanning (see below).

MRI In Vivo

After animal preparation, an anatomical T2-weighted scout image was obtained using a multislice fast spin-echo sequence with a 25×25 mm field of view (FOV), 128×128 image matrix, TR=4 s, TE=15 ms, and 8 echoes with k-space centered on the 4th echo. To determine the distribution and preliminary pharmacokinetic behavior, MR images were obtained using T2* weighted gradient echo MRI. Gradient echo images were acquired using a 25×25 mm FOV over a 64×64 matrix, in multiple 1 mm axial slices which covered the entire region of the tumor. Pre-IV-injection scans were obtained with (i) TR=80 ms/TE=7.5 ms and (ii) TR=2 s/TE=15 ms. During IV injection of the nanoparticle preparation, a dynamic gradient-echo sequence with a time resolution of 10 s was used to characterize the uptake of the nanoparticle into normal tissue and the tumor over 7 minutes. Post-IV-injection gradient-echo scans were then acquired over 2 hours to quantify clearance of nanoparticle contrast. In cases where contrast persisted, images were acquired at later timepoints, until contrast returned to the baseline level or reached equilibrium.

Images were analyzed by measuring signal intensity time courses within manually drawn ROIs in vessel, normal brain, and tumor. Relative concentration of the NP contrast agents was derived from the signal intensity:
Relative concentration ∝ΔR₂*=−log(S/S₀)/TE [Equation 1]
where S is signal intensity following administration of the contrast agent, S₀ is the initial signal intensity and TE is the echo time. For the nanoparticle uptake relative concentration timecourse, the minimum signal-to-noise ratio (SNR) value was calculated for each tissue type, as well as the time to minimum SNR. For the extended timecourse of relative concentration (nanoparticle clearance), exponential decays were fitted to the relative concentration data and half-lives derived for clearance of the nanoparticles into/from vasculature, contralateral brain and tumor tissue.
Results

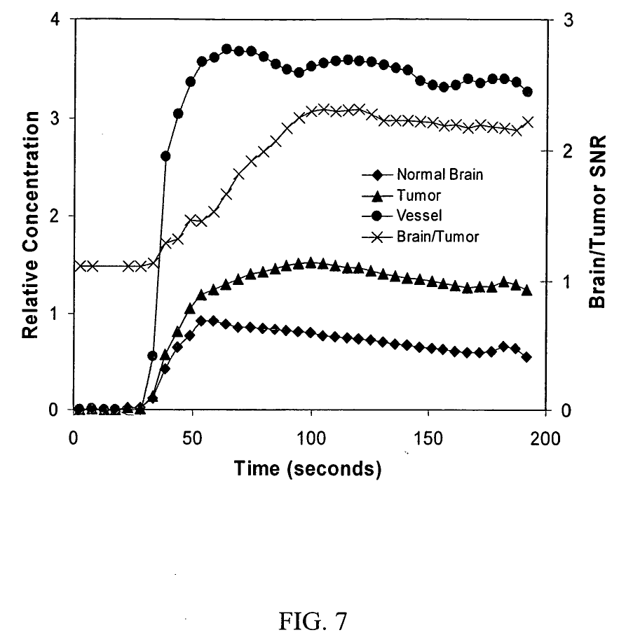

Uptake results are represented in FIG. 7.

TABLE 1
Baseline and minimum SNR data for normal brain, tumor and
vasculature.

| | | | Time to | SNR |
|---|---|---|---|---|
| | Baseline | Minimum | Minimum SNR | Decrease |
| Tissue Type | SNR | SNR | (min) | (%) |
| Normal Brain | 36.5 | 15 | 56 | 59 |
| Tumor | 33 | 7 | 144 | 79 |
| Vessel | 70 | 2 | 63 | 97 |

Brain/Tumor SNR Ratio

Maximum: 2.3

Time to maximum: 113

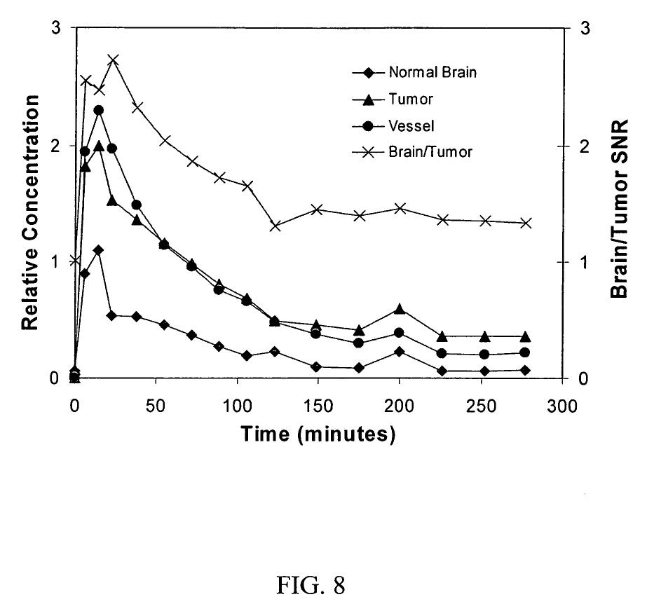

Clearance results are shown in FIG. 8.

TABLE 2
Nanoparticle half-life and equilibrium signal (as % of initial signal) in
vasculature, normal brain and tumor tissue, calculated from gradient echo
timecourse data with TR = 80 ms/TE = 7.5 ms.

| | | Equilibrium |
|---|---|---|
| Tissue Type | Half-life (minutes) | SNR/Initial SNR (%) |
| Vessel | 46 ± 5 | 68 ± 6 |
| Normal Brain | 33 ± 3 | 96 ± 10 |
| Tumor | 38 ± 3 | 85 ± 4 |

TABLE 3
Nanoparticle half-life and equilibrium signal (as % of initial signal) in
vasculature, normal brain and tumor tissue, calculated from gradient echo
timecourse data with TR = 2 s/TE = 15 ms.

| | | Equilibrium |
|---|---|---|
| Tissue Type | Half-life (minutes) | SNR/Initial SNR (%) |
| Vessel | 38 ± 2 | 81 ± 10 |
| Normal Brain | 28 ± 8 | 90 ± 39 |
| Tumor | 40 ± 5 | 70 ± 10 |

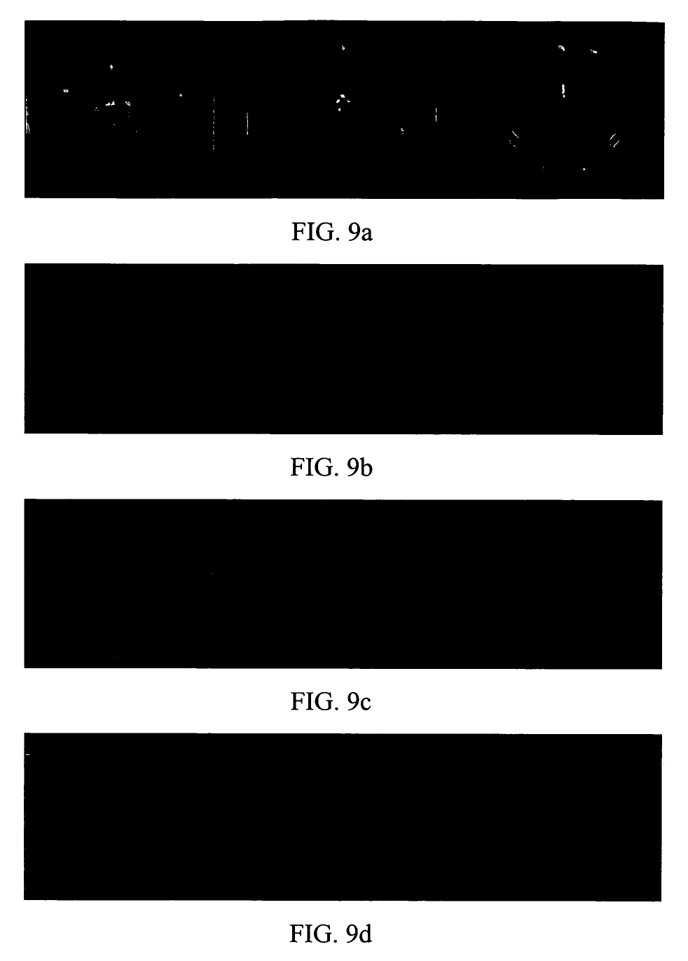

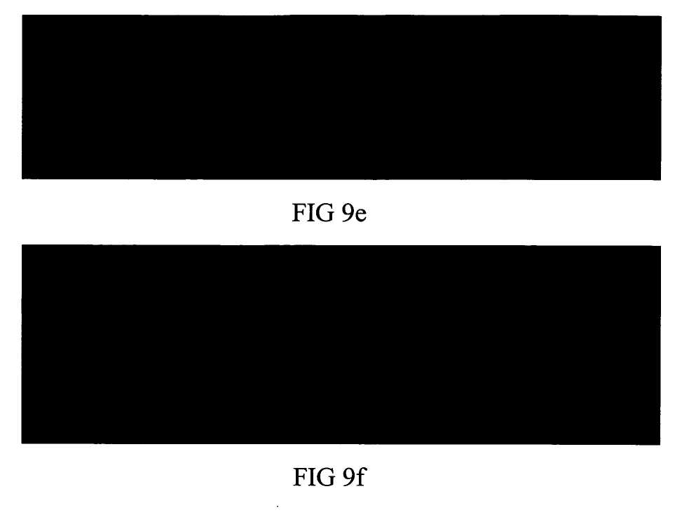

FIGS. 9a-9f represent spin-echo anatomical scout images, and example pre- and post-nanoparticle injection gradient-echo images at various time points (TR=2 s/TE=15 ms).

The brain/tumor contrast induced by this iron-oxide nanoparticle was high, reaching a maximum B/T SNR of 2.3 at 113 s during uptake. The contrast uptake was equally rapid in the vasculature and normal brain, with clearance occurring immediately following the signal minimum. In the tumor, the uptake (signal minimum and onset of clearance) was delayed compared with that of the normal brain and vasculature. This might be a consequence of subtle cumulative effects of tumor heterogeneity, blood flow, and permeability. Though the equilibrium t_{1/2} for vasculature, normal brain and tumor clearance are roughly similar (range 33-46 minutes), this nanoparticle exhibited an apparent biphasic clearance. In the tumor, the contrast induced by this particle never completely cleared, as evidenced by the equilibrium signal level which was 70-80% of the initial signal level. The normal brain signal did return to baseline. Indeed, continued imaging of the tumor showed evidence of continued tumor growth and contrast enhancement at 5 days (the time the animals were euthanized). These data suggest selective uptake of this nanoparticle into the tumor cells, and retention of the nanoparticle for at least five days.

### EXAMPLE 12

Synthesis of Targeted Nanoparticles

Synthesis of Fluorescently-Labeled Sorbitol Nanoparticles Conjugated to the F3-Peptide

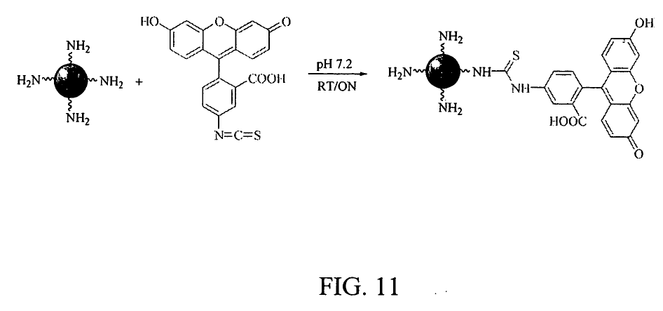

FIG. 11 shows the synthesis of FITC-Sorbitol nanoparticle conjugates according to one embodiment of the present invention. A 20 mL reaction vial was charged with amine functionalized sorbitol nanoparticles (500 mg) in sodium phosphate buffer (0.1 M, pH 7.2) and sodium chloride (0.15 M) solution. The mixture was treated with fluorescein isothiocyanate (FITC, 5 mg, Pierce) and the mixture was sonicated for 5 minutes at RT. The reaction mixture was protected from light and gently stirred at RT overnight. The mixture was transferred to an Amicon stirred cell (50 mL) equipped with a magnetic stirrer and thoroughly washed with water.

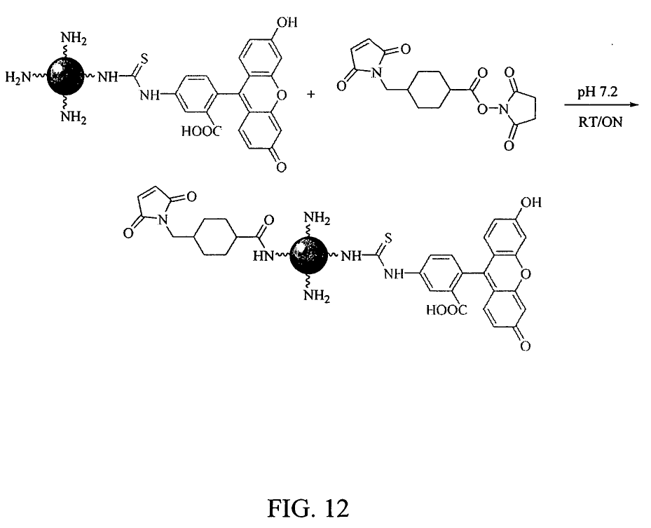

Conjugation of sulfo-SMCC to the fluorescently-labeled nanoparticles (shown in FIG. 12) was accomplished by diluting the FITC-labeled nanoparticles with sodium phosphate buffer (0.1 M, pH 7.2), covering with aluminum foil and was purging with a constant flow of argon. The solution was treated with sulfo-SMCC (30 mg, 0.07 mmol) and the mixture was stirred overnight at RT under argon. The reaction mixture was thoroughly washed with argon purged water in an Amicon stirred cell equipped with a magnetic stirrer.

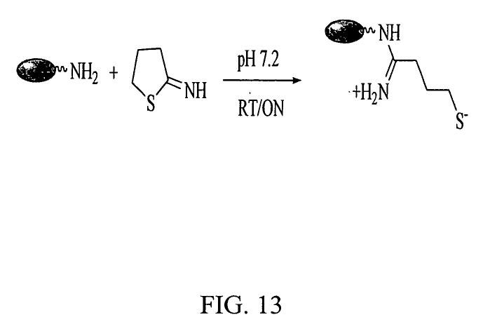

The F3-peptide-2-iminothiolane conjugate was prepared as shown in FIG. 13. A 20 mL glass reaction vial was charged with F3-peptide (20 mg, 58 μmol) in sodium phosphate buffer (0.1 M, pH 7.2) and was treated with 2-iminothiolane (3 mg, 2-IT). The mixture was gently agitated for 30 min at RT followed by 2 h at 4° C.

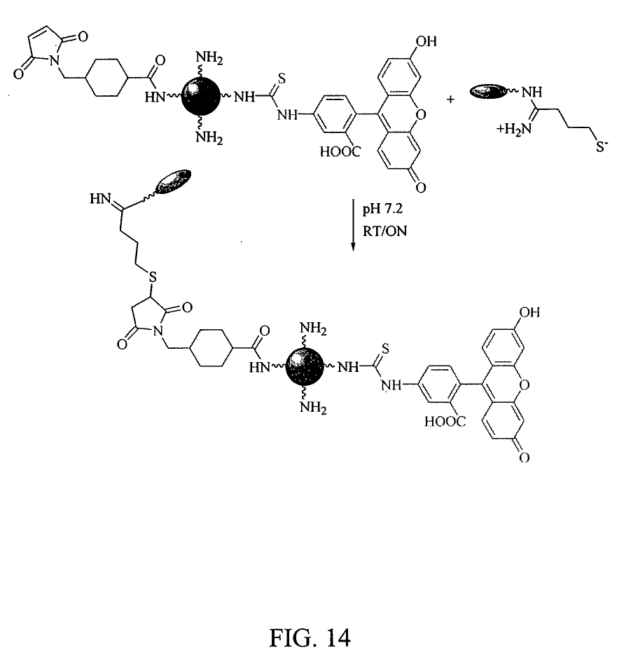

The FITC-SMCC conjugated nanoparticles (125 mg) in sodium phosphate buffer (0.1 M, pH 7.2) were treated with the F3-peptide-2-IT conjugate as shown in FIG. 14 and the mixture was gently agitated at RT overnight. The reaction mixture was thoroughly washed with water in an Amicon stirred cell equipped with a magnetic stirrer. The concentrated F3-peptide conjugated fluorescent labeled nanoparticles solution was stored at 4° C. until further use.

### EXAMPLE 13

Synthesis of Fluorescent Labeled Fe₃O₄-Encapsulated Sorbitol Nanoparticle Conjugated to the F3-Peptide

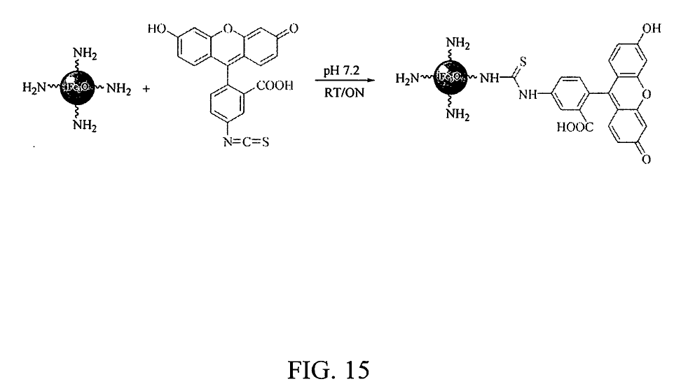

Synthesis of FITC-Fe₃O₄-Sorbitol nanoparticle conjugates are shown in FIG. 15. A 20 mL reaction vial was charged with amine functionalized sorbitol nanoparticles (500 mg) in sodium phosphate buffer (0.1 M, pH 7.2) and sodium chloride (0.15 M) solution. The mixture was treated with FITC (5 mg, Pierce) and the mixture was sonicated for 5 minutes at RT. The reaction mixture was protected from light and gently stirred at RT overnight. The mixture was transferred to an Amicon stirred cell (50 mL) equipped with a magnetic stirrer and thoroughly washed with water.

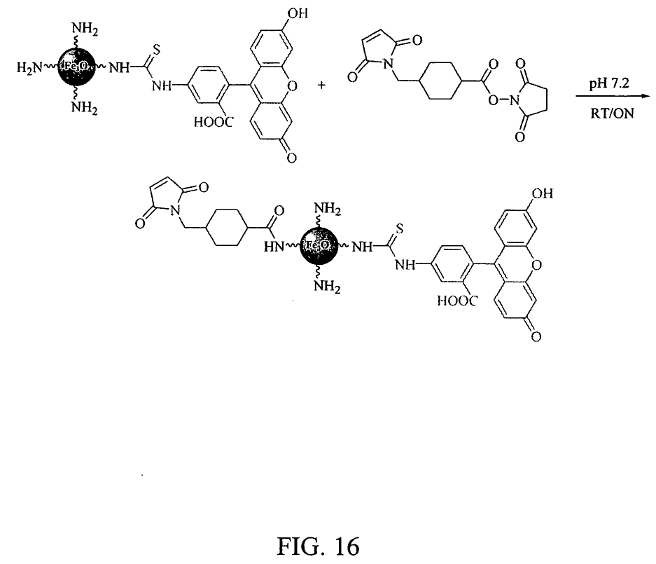

Conjugation of sulfo-SMCC to the fluorescent labeled Fe₃O₄-nanoparticles is shown in FIG. 16. The fluorescent labeled Fe₃O₄ nanoparticle solution was diluted with sodium phosphate buffer (0.1 M, pH 7.2), covered with aluminum foil and purged with a constant flow of argon. The solution was treated with sulfo-SMCC (30 mg, 0.07 mmol) and the mixture was stirred overnight at RT under argon. The reaction mixture was thoroughly washed with argon purged water in an Amicon stirred cell equipped with a magnetic stirrer.

A 20 mL glass reaction vial was charged with F3-peptide (20 mg, 58 μmol) in sodium phosphate buffer (0.1 M, pH 7.2) and was treated with 2-iminothiolane (3 mg, 2-IT). The mixture was gently agitated for 30 min at RT followed by 2 h at 4° C.

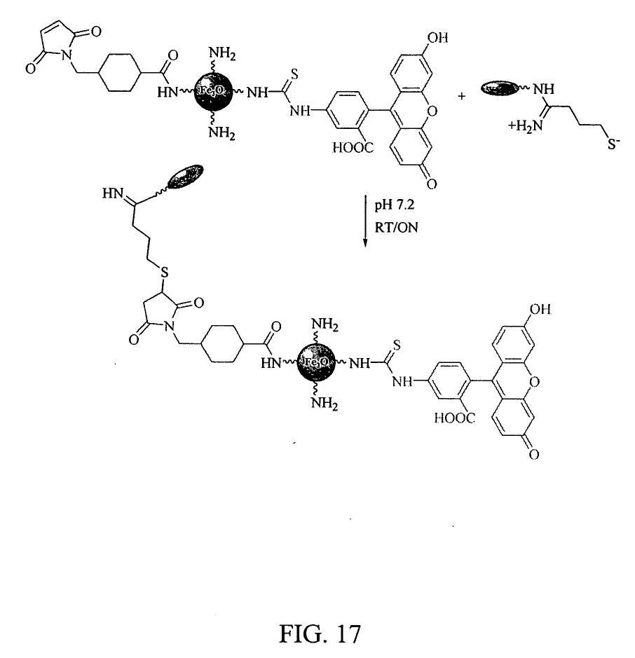

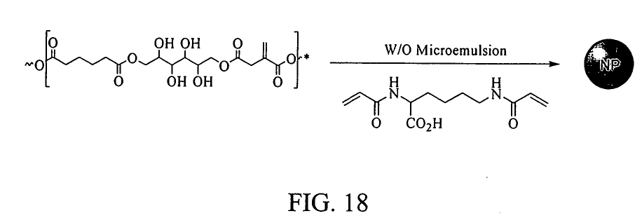

The FITC-SMCC conjugated nanoparticles (125 mg) in sodium phosphate buffer (0.1 M, pH 7.2) were treated with the F3-peptide-2-IT conjugate as shown in FIG. 17 and the mixture was gently agitated at RT overnight. The reaction mixture was thoroughly washed with water in an Amicon stirred cell equipped with a magnetic stirrer. The concentrated F3-peptide conjugated fluorescent labeled nanoparticles solution was stored at 4° C. until further use.

Degradation of Sorbitol Nanoparticles

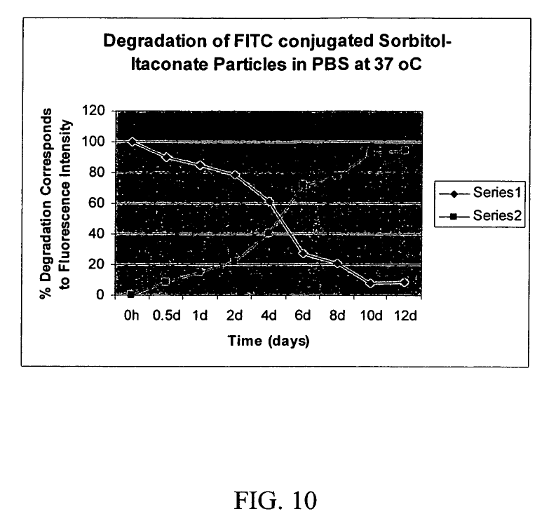

Amine functionalized blank nanoparticles were treated with an excess of FITC in phosphate buffer and stirred overnight at RT under argon. They were then washed thoroughly with water and added to plasma (5 mL). The solution was incubated at 37° C. and samples were collected at intervals and was frozen at −20° C. Similarly the degradation was done in PBS (pH 7.4) at 37° C. and the samples were frozen at −20° C. The results are demonstrated in FIG. 10, Series 1 shows the decrease in fluorescence intensity in the original particle over time and series 2 shows the increase in fluorescence intensity in the filtrate over time.

### EXAMPLE 14

Synthesis of Sorbitol-Itaconic Acid-Glycolic Acid Polyester.

Into a 100 mL round bottom flask was transferred sorbitol (9.10 g, 50 mmol) glycolic acid (0.78 g, 10 mmol) and itaconic acid (6.5 g, 50 mmol). The reactants were heated with stirring to 140° C. and the mixture melted. The temperature of the reaction mixture was then lowered to 90-95° C. and the reaction components remained as a viscous liquid. Then, Novozym®-435 beads (10% wt/wt relative to monomers, 1.6 g, dried at 25° C./10 mmHg/24 hrs) were added. Within 2 hrs the reaction mixture appeared monophasic with suspended catalyst beads. The flask was sealed with a rubber septum and the reaction was maintained at 90° C. with mixing. After the first 6 hrs of the reaction, the contents of the reaction were maintained under reduced pressure (40 mmHg). The polymerization was terminated after 48 hrs by dissolving the reaction mixture in methanol, removing the enzyme by filtration, and stripping off the solvent in vacuo. The product was then dried in a vacuum oven (10 mmHg, 30° C., 24 hrs).

### EXAMPLE 15

Synthesis of Glycerol-Itaconic Acid Polyester

Into a 100 mL round bottom flask was transferred glycerol (2.30 g, 25 mmol) and itaconic acid (3.25 g, 25 mmol). The reactants were heated with stirring to 140° C. and the mixture melted. The temperature of the reaction mixture was then lowered to 90-95° C. and the reaction components remained as a viscous liquid. Then, NOVOZYM®)-435 beads (10% wt/wt relative to monomers, 555 mg, dried at 25° C./10 mmHg/24 hrs) were added. Within 2 hrs the reaction mixture appeared monophasic with suspended catalyst beads. The flask was sealed with a rubber septum and the reaction was maintained at 90° C. with mixing. After the first 6 hrs of the reaction, the contents of the reaction were maintained under reduced pressure (40 mmHg). The polymerization was terminated after 48 hrs by dissolving the reaction mixture in methanol, removing the enzyme by filtration, and stripping off the solvent in vacuo. The product was then dried in a vacuum oven (10 mmHg, 30° C., 24 hrs).

### EXAMPLE 16

Synthesis of Sorbitol-Itaconic Acid-Adipic Acid Polyester.

Into a 100 mL round bottom flask was transferred sorbitol (9.1 g, 50 mmol), itaconic acid (3.25 g, 25 mmol) and adipic acid (3.65 g, 25 mmol). The reactants were heated with stirring to 150° C. and the mixture melted. The temperature of the reaction mixture was then lowered to 90-95° C. and the reaction components remained as a viscous liquid. Then, Novozym®-435 beads (10% wt/wt relative to monomers, 1.6 g, dried at 25° C./10 mmHg/24 hrs) were added. Within 2 hrs the reaction mixture appeared monophasic with suspended catalyst beads. The flask was sealed with a rubber septum and the reaction was maintained at 90° C. with mixing. After the first 6 hrs of the reaction, the contents of the reaction were maintained under reduced pressure (40 mmHg). The polymerization was terminated after 48 hrs by dissolving the reaction mixture in methanol, removing the enzyme by filtration, and stripping off the solvent in vacuo. The product was then dried in a vacuum oven (10 mmHg, 30° C., 24 hrs).

### EXAMPLE 17

Synthesis of PEG-Sorbitol-Itaconic Acid Polyester

Into a 100 mL round bottom flask was transferred sorbitol (9.1 g, 50 mmol), polyethylene glycol dicarboxylate (Ave MW 600, 15 g, 25 mmol) and itaconic acid (3.25 g, 25 mmol). The reactants were heated with stirring to 130° C. and the mixture melted. The temperature of the reaction mixture was then lowered to 90-95° C. and the reaction components remained as a viscous liquid. Then, NOVOZYM®-435 beads (10% wt/wt relative to monomers, 2.75 g, dried at 25° C./10 mmHg/24 hrs) were added. Within 2 hrs the reaction mixture appeared monophasic with suspended catalyst beads. The flask was sealed with a rubber septum and the reaction was maintained at 90° C. with mixing. After the first 6 hrs of the reaction, the contents of the reaction were maintained under reduced pressure (40 mmHg). The polymerization was terminated after 48 hrs by dissolving the reaction mixture in methanol, removing the enzyme by filtration, and stripping off the solvent in vacuo. The product was then dried in a vacuum oven (10 mmHg, 30° C., 24 hrs).

### EXAMPLE 18

Synthesis of Sorbitol-Acrylamidoglycolic Acid Polyester

Into a 100 mL round bottom flask was transferred sorbitol (1.82 g, 10 mmol), acrylamidoglycolic acid (363 mg, 2.5 mmol) and adipic acid (1.74 g, 10 mmol). The reactants were heated with stirring to 160° C. and the mixture melted. The temperature of the reaction mixture was then lowered to 90-95° C. and the reaction components remained as a viscous liquid. Then, NOVOZYM®-435 beads (10% wt/wt relative to monomers, 400 mg, dried at 25° C./10 mmHg/24 hrs) were added. Within 2 hrs the reaction mixture appeared monophasic with suspended catalyst beads. The flask was sealed with a rubber septum and the reaction was maintained at 90° C. with mixing. After the first 6 hrs of the reaction, the contents of the reaction were maintained under reduced pressure (40 mmHg). The polymerization was terminated after 48 hrs by dissolving the reaction mixture in methanol, removing the enzyme by filtration, and stripping off the solvent in vacuo. The product was then dried in a vacuum oven (10 mmHg, 30° C., 24 hrs).

### EXAMPLE 19

Synthesis of Sorbitol-Acrylamidoglutamic Acid Polyester

Into a 100 mL round bottom flask was transferred sorbitol (1.82 g, 10 mmol), acrylamidoglutamic acid (503 mg, 2.5 mmol) and adipic acid (1.74 g, 10 mmol). The reactants were heated with stirring to 160° C. and the mixture melted. The temperature of the reaction mixture was then lowered to 90-95° C. and the reaction components remained as a viscous liquid. Then, NOVOZYM®-435 beads (10% wt/wt relative to monomers, 400 mg, dried at 25° C./10 mmHg/24 hrs) were added. Within 2 hrs the reaction mixture appeared monophasic with suspended catalyst beads. The flask was sealed with a rubber septum and the reaction was maintained at 90° C. with mixing. Furthermore, after the first 6 hrs of the reaction, the contents of the reaction were maintained under reduced pressure (40 mmHg). The polymerization was terminated after 48 hrs by dissolving the reaction mixture in methanol, removing the enzyme by filtration, and stripping off the solvent in vacuo. The product was then dried in a vacuum oven (10 mmHg, 30° C., 24 hrs).

### EXAMPLE 20

Synthesis of Sorbitol Diitaconate (SDI):

Sorbitol (1.82 g, 10 mmol) and itaconic anhydride (2.24 g, 20 mmol) were melted together at 110° C. under argon and the mixture was heated at 100° C. for 24 hrs. The thick oil obtained was dissolved in methanol (10 mL) and precipitated in ether (100 mL). The product obtained was directly used for nanoparticle synthesis.

### EXAMPLE 21

Synthesis of Glycerol Diitaconate

Glycerol (0.92 g, 10 mmol) and itaconic anhydride (2.24 g, 20 mmol) were melted together at 100° C. under argon and the mixture was heated at 80° C. for 24 hrs. The thick oil obtained was dissolved in methanol (10 mL) and precipitated in ether (100 mL). The product obtained was directly used for nanoparticle synthesis.

### EXAMPLE 22

Synthesis of Propylene Glycol Diitaconate

Propylene glycol (0.76 g, 10 mmol) and itaconic anhydride (2.24 g, 20 mmol) were melted together at 80° C. under argon and the mixture was heated at 60° C. for 24 h. The thick oil obtained was dissolved in methanol (10 mL) and precipitated in ether (100 mL). The product obtained was directly used for nanoparticle synthesis.

### EXAMPLE 23

Synthesis of Ethylene Glycol Diitaconate

Ethylene glycol (0.62 g, 10 mmol) and itaconic anhydride (2.24 g, 20 mmol) were melted together at 80° C. under argon and the mixture was heated at 60° C. for 24 h. The thick oil obtained was dissolved in methanol (10 mL) and precipitated in ether (100 mL). The product obtained was directly used for nanoparticle synthesis.

### EXAMPLE 24

Synthesis of Blank Sorbitol-Itaconate (PSIA) Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and was dissolved in 0.1 M sodium phosphate buffer (3.5 mL, pH 7.4) by sonication until a uniform solution resulted. The uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 hrs under argon. Hexane was removed under reduced pressure to give a thick syrupy residue which was diluted with ethanol (100 mL). The mixture was sonicated and the separated particles washed in an Amicon stirred cell (500K cut-off filter, Millipore, 200 mL) with ethanol (5×150 mL). The white nanoparticles obtained were dried under nitrogen and gently crushed to a fine powder (2.0 g). The material was stored at 4° C. Similarly, blank nanoparticles were synthesized using all other polymers.

### EXAMPLE 25

Synthesis of Blank Sorbitol-Itaconate-Sorbitol Diitaconate (PSIA-SDI) Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g, 25 wt %) was dissolved separately in 0.1 M solution of sodium phosphate buffer (1.0 mL, pH 7.4). The PSIA and SDI solution were mixed together and sonicated until a clear uniform solution was obtained. The uniform suspension was added to the hexane reaction mixture and was stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500 K cut-off filter, Millipore, 200 mL) with ethanol (5×150 mL). The white nanoparticles obtained were dried under nitrogen and gently crushed to a fine powder (2.5 g). The material was stored at 4° C.

Similarly, blank cross-linked nanoparticles were synthesized using all other polymers and cross linking agents, e.g., ethylene glycol diitaconate and propylene glycol diitaconate with 15, 25 and 35 wt %.

### EXAMPLE 26

Synthesis of Fluorescein Encapsulated PSIA Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. The PSIA and 6-carboxyfluorescein (25 mg) were mixed together and sonicated until a clear uniform solution was obtained. The deep yellow uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles washed in an Amicon stirred cell (500 K cut-off filter, Millipore, 200 mL) with ethanol (5×150 mL). The white nanoparticles obtained were dried under nitrogen and gently crushed to a fine powder (2.02 g). The material was stored at 4° C.

### EXAMPLE 27

Synthesis of Fe₃O₄ (W11) Encapsulated PSIA Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. The PSIA and Fe₃O₄ (2.0 mL) solution were mixed together and sonicated until a clear dark brown uniform solution was obtained. The dark brown colored uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500 K cut-off filter, Millipore, 200 mL) with ethanol (5×150 mL). The white nanoparticles obtained were dried under nitrogen and gently crushed to a fine powder (2.11 g). The material was stored at 4° C.

### EXAMPLE 28

Synthesis of Fe₃O₄ (EMG 805) Encapsulated PSIA Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. The PSIA and Fe₃O₄ (2.0 mL) solution were mixed together and sonicated until a clear uniform solution was obtained. The dark brown colored uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500K cut-off filter, Millipore, 200 mL) with ethanol (5×150 mL). The white nanoparticles obtained were dried under nitrogen and gently crushed to a fine powder (2.02 g). The material was stored at 4° C.

### EXAMPLE 29

Synthesis of Fluorescein Encapsulated PSIA-SDI Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g, 25 wt %) and 6-carboxyfluorescein were dissolved in 0.1 M solution of sodium phosphate buffer (1.0 mL, pH 7.4). The PSIA and SDI solution were mixed together and sonicated until a clear uniform solution was obtained. The deep yellow uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles washed in an Amicon stirred cell (500 K cut-off filter, Millipore, 200 mL) with ethanol (5×150 mL). The white nanoparticles obtained were dried under nitrogen and gently crushed to a fine powder (2.52 g). The material was stored at 4° C.

### EXAMPLE 30

Synthesis of Fe₃O₄ (W11) Encapsulated PSIA-SDI Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g, 25 wt %) and Fe₃O₄ (2.5 mL) solution were dissolved in 0.1 M solution of sodium phosphate buffer (1.0 mL, pH 7.4). The PSIA and SDI solution were mixed together and sonicated until a clear uniform solution was obtained. The dark brown colored uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles washed in an Amicon stirred cell (500 K cut-off filter, Millipore, 200 mL) with ethanol (5×150 mL). The white nanoparticles obtained were dried under nitrogen and gently crushed to a fine powder (2.62 g). The material was stored at 4° C.

### EXAMPLE 31

Synthesis of Fe₃O₄ (EMG 805) Encapsulated PSIA-SDI Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g) and Fe₃O₄ (2.5 mL) solution were dissolved in water 0.1 M solution of sodium phosphate buffer (1.0 mL, pH 7.4). The PSIA and SDI solution were mixed together and sonicated until a clear uniform solution was obtained. The dark brown colored uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500K cut-off filter, Millipore, 200 mL) with ethanol (5×150 mL). The white nanoparticles obtained were dried under nitrogen and gently crushed to a fine powder (2.64 g). The material was stored at 4° C.

### EXAMPLE 32

Synthesis of Gemcitabine Encapsulated PSIA-SDI Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g) and gemcitabine hydrochloride (25 mg) were dissolved in 0.1 M solution of sodium phosphate buffer (1.0 mL, pH 7.4). The PSIA and SDI solution were mixed together and sonicated until a clear uniform solution was obtained. The clear uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500 K cut-off filter, Millipore, 200 mL) with ethanol (5×150 mL). The white nanoparticles obtained were dried under nitrogen and gently crushed to a fine powder (2.53 g). The material was stored at 4° C.

### EXAMPLE 33

Synthesis of Doxorubicin Encapsulated PSIA-SDI Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g) and doxorubicin (25 mg) were dissolved in 0.1 M solution of sodium phosphate buffer (1.0 mL, pH 7.4). The PSIA and SDI solution was mixed together and sonicated until a clear uniform solution was obtained. The dark red colored uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500 K cut-off filter, Millipore, 200 mL) with ethanol (5×150 mL). The white nanoparticles obtained were dried under nitrogen and gently crushed to a fine powder (2.54 g). The material was stored at 4° C.

### EXAMPLE 34

Synthesis of TAXOL® Encapsulated PSIA-SDI Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g) was dissolved in 0.1 M solution of sodium phosphate buffer (1.0 mL, pH 7.4). The PSIA and SDI solution were mixed together and sonicated until a clear uniform solution was obtained. TAXOL® was dissolved in acetonitrile (2 mL) and mixed with the monomer solution and sonicated to get a milky clear solution. The uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500 K cut-off filter, Millipore, 200 mL) with ethanol (5×150 mL). The white nanoparticles obtained were dried under nitrogen and gently crushed to a fine powder (2.24 g). The material was stored at 4° C.

### EXAMPLE 35

Synthesis of Cytidine Encapsulated PSIA-SDI Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g) and cytidine (25 mg) were dissolved in 0.1 M solution of sodium phosphate buffer (1.0 mL, pH 7.4). The PSIA and SDI solution were mixed together and sonicated until a clear uniform solution was obtained. The clear uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500 K cut-off filter, Millipore, 200 mL) with ethanol (5×150 mL). The white nanoparticles obtained were dried under nitrogen and gently crushed to a fine powder (2.46 g). The material was stored at 4° C.

### EXAMPLE 36

Synthesis of Ru Encapsulated PSIA Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g) and Ru dye (˜25 mg) were dissolved in 0.1 M solution of sodium phosphate buffer (1.0 mL, pH 7.4). The PSIA and SDI solution were mixed together and sonicated until a clear red uniform solution was obtained. The deep red uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500K cut-off filter, Millipore, 200 mL) with ethanol (5×150 mL). The white nanoparticles obtained were dried under nitrogen and gently crushed to a fine powder (2.59 g). The material was stored at 4° C.

### EXAMPLE 37

Synthesis of Photophrin Encapsulated PSIA Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g) and photophrin (25 mg) were dissolved in a 0.1 M solution of sodium phosphate buffer (1.0 mL, pH 7.4). The PSIA and SDI solution were mixed together and sonicated until a clear dark brown uniform solution was obtained. The black uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles washed in an Amicon stirred cell (500 K cut-off filter, Millipore, 200 mL) with ethanol (5×150 mL). The white nanoparticles obtained were dried under nitrogen and gently crushed to a fine powder (2.54 g). The material was stored at 4° C.

### EXAMPLE 38

Synthesis of Phthalocyanine Encapsulated PSIA Nanoparticles

An oven-dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. A 20 mL glass sample tube was charged with PSIA (2.0 g) and dissolved in 0.1 M sodium phosphate buffer (3.5 mL, pH 7.4) by sonication until a uniform solution was resulted. SDI (0.5 g) and phthalocyanine (25 mg) were dissolved in 0.1 M solution of sodium phosphate buffer (1.0 mL, pH 7.4). The PSIA and SDI solution were mixed together and sonicated until a clear blue uniform solution was obtained. The deep blue uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500 K cut-off filter, Millipore, 200 mL) with ethanol (5×150 mL). The white nanoparticles obtained were dried under nitrogen and gently crushed to a fine powder (2.55 g). The material was stored at 4° C.

### EXAMPLE 39

Synthesis of Bromocresol Green Encapsulated PSIA Nanoparticles

An over dried 250 mL round bottom flask was charged with hexane (100 mL) and stirred under a constant purge of argon. Aerosol OT (3.40 g) and Brij 30 (6.5 mL) were added to the reaction flask and stirring was continued under argon until a uniform solution was formed. In the mean time, a 20 mL glass sample tube was charged with PSIA (2.0 g) and dissolved in 0.1 M sodium phosphate buffer (3.5 mL, pH 7.4) by sonication until a uniform solution resulted. SDI (0.5 g) and bromocresol green (25 mg) were dissolved in 0.1 M solution of sodium phosphate buffer (1.0 mL, pH 7.4). The PSIA and SDI solution were mixed together and sonicated until a clear orange uniform solution was obtained. The deep orange uniform suspension was added to the hexane reaction mixture and stirred vigorously for 15 minutes at room temperature under argon. Ammonium persulfate (10% solution in water, 0.065 mL) and TEMED (0.085 mL) were added to the reaction mixture as polymerization initiator. The reaction mixture was stirred vigorously at room temperature for 15 h under argon. Hexane was removed under reduced pressure to give a thick syrupy residue which was diluted with ethanol (100 mL). The mixture was sonicated and the separated particles were washed in an Amicon stirred cell (500 K cut-off filter, Millipore, 200 mL) with ethanol (5×150 mL). The white nanoparticles obtained were dried under nitrogen and gently crushed to a fine powder (2.52 g). The material was stored at 4° C.

### EXAMPLE 40

Degradation of Ru-Dye Encapsulated Sorbitol Nanoparticles with NaOH

Ru-dye containing nanoparticles (200 mg) were dissolved in 1 N sodium hydroxide solution and filtered through 100 K cutoff membrane and the filtrate and original solution were monitored at 1 h, 12 h and 36 h by UV spectroscopy. The amount of dye coming out in the filtrate indicated the percentage of degradation over time. FIG. 5 shows the percent degradation over the time period 0-36 hours. Series 1 shows the amount of dye released in each measurement and series 2 indicates the total amount of dye released over time.

### EXAMPLE 41

Degradation of Sorbitol Particle by Sizing at 37° C. in PBS (pH 7.4)

Sorbitol nanoparticles (200 mg) were dissolved in water (10 mL) by sonication and were filtered through 0.8, 0.45 and 0.2 μm filters respectively. The solution was washed in a 50 mL Amicon stirred cell with water (10×10 mL) and then with PBS (1 X, 3×10 mL). Particle size was measured. The solution was incubated at 37° C. with shaking and the particle size was monitored every 24 hrs. Table 4 shows the particle size distribution by intensity, volume and number weighted measurements.

TABLE 4

| | Mean diameter (nm) | | | |
|---|---|---|---|---|
| | | | Size % of | |
| Particle | Time | Intensity | original | Number |
| Specifications | (hours) | weighted | Particles | weighted |
| Blank acidic particles | 0 | 136.5 | 100 | 33.3 |
| | 20 | 96.5 | 70.6 | 27.0 |
| | 40 | 86.7 | 63.5 | 17.8 |
| | 60 | 80.9 | 59.3 | 21.6 |
| | 80 | 76.8 | 56.3 | 15.8 |
| | 100 | 75.9 | 55.6 | 18.3 |
| | 120 | 74.4 | 54.5 | 13.5 |
| | 140 | 68.7 | 50.3 | 14.7 |
| | 160 | 64.6 | 47.3 | |
| | 200 | 53.2 | 38.9 | |
| | 12 days | 28.2 | 20.7 | |
| | 15 days | No | 0.0 | 0.0 |
| | | particle | | |
| | | detected | | |

### EXAMPLE 42

Degradation of Fluorescein Conjugated Sorbitol Nanoparticles Incubated at 37° C. in PBS (pH 7.4) Followed by Sizing

Fluorescein conjugated sorbitol nanoparticles (200 mg) were dissolved in water and incubated at 37° C. with shaking and the samples were taken out at regular intervals and filtered through Centricon. The filtrate and original particles were measured for fluorescence intensity. FIG. 10, series 1 shows the decrease in fluorescence intensity of the original particle and series 2 shows the increase in fluorescence intensity of the filtrate over time.

### EXAMPLE 43

Synthesis of Nanoparticles from Sorbitol-Sebacic Acid-Itaconic Acid Polymer

Into a 100 mL round bottom flask was added sorbitol (2 mmol), sebacic acid (1 mmol) and itaconic acid (1 mmol). The reactants were heated with stirring at 115° C. and the mixture melted. The temperature of the reaction mixture was then lowered to 90° C. and the reaction components remained as a viscous liquid. Then, Novozym®-435 beads (10% wt/wt relative to monomers, dried at 25° C./10 mmHg/24 hrs) were added. Within 15 minutes the reaction mixture appeared monophasic with suspended catalyst beads. The flask was sealed with a rubber septum and the reaction was maintained at 90° C. with mixing. After the first 2 hrs of the reaction, the contents of the reaction were maintained under reduced pressure (40 mmHg). The polymerization was terminated at 48 h by dissolving the reaction mixture in excess methanol, removing the enzyme by filtration and stripping the solvent in vacuo. The product was then dried in a vacuum oven (RT, 24h).

Blank nanoparticles were synthesized from Sorb:SA:IA polymer with 25% cross linking of ethyleneglycol diitaconate (EGDI).

### EXAMPLE 44

Synthesis of Nanoparticles Comprising a Water-Soluble, Lysine-Diacrylamide Cross Linker

A 20 mL scintillation vial was charged with sorbitol-itaconic acid-adipic acid polyester (1.4 g), lysine-diacrylamide (0.5 g), and ddI-H₂O (8.0 mL). The resulting mixture was sonicated for 5 min to effect complete dissolution to a clear homogenic solution. AOT (5.2 g) and Brij 30 (6.4 mL) were dissolved in hexane (130 mL) in a 250 mL round bottom flask under an argon atmosphere. The hexane solution was stirred for 20 min with argon bubbling. The aqueous solution in the scintillation vial was then added to the hexane solution and the resulting clear monophasic solution was stirred for 20 min under argon. Radical polymerization was initiated by adding Ammonium Persulfate (130 μL, 10%) and TEMED (180 μL). The mixture was stirred overnight and hexane was removed by rotovap. Ethanol was added to the resulting thick syrupy solution and the precipitate was filtered through an Amicon stirred cell (250 mL) equipped with 100 kD MWCO filter. The precipitate was washed with ethanol (4×100 mL) and dried to yield 750 mg.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A process for producing a polymeric nanoparticle comprising:
(a) condensing one or more primary dihydroxy compounds and one or more diacids to generate a polyester;
(b) adding one or more cross-linkers selected from the group consisting of ethylene glycol diitaconate, glycerol (bis) itaconate, sorbitol diitaconate, glycerol dimethacrylate and divinyl citrate;
(c) initiating polymerization to generate a solid particle; and
(d) removing the solid particle from solution.

2. The process of claim 1, wherein said condensing occurs via esterification.

3. The process of claim 1, wherein said condensing occurs via enzyme catalysis.

4. The process of claim 3, wherein the enzyme is a lipase.

5. The process of claim 1, wherein said initiating in (c) occurs in the presence of one or more surfactants.

6. The process of claim 1, further comprising passing the removed solid particle through one or more porous filters to generate a nanoparticle that is less than about 200 nm in diameter.

7. The process of claim 1, further comprising adding an agent to be encapsulated to the solution prior to said initiation (c).

8. The process of claim 1, further comprising adding in (a) a functionalized monomer, thereby generating a functionalized group on the surface of the nanoparticle.

9. A polymeric nanoparticle produced by the process of claim 1.

10. The polymeric nanoparticle of claim 9, wherein said nanoparticle is biodegradable.

11. The polymeric nanoparticle of claim 9, wherein said primary dihydroxy compound is selected from the group consisting of sorbitol, mannitol, iditol, sucrose, fructose, lactose, ribose, maltose, glycerol, ethylene glycol, propylene glycol and glycerol.

12. The polymeric nanoparticle of claim 9, wherein said diacid is selected from the group consisting of itaconic acid, adipic acid, succinic acid, fumaric acid, and acylamidoglutamic acid.

13. The polymeric nanoparticle of claim 9, further comprising a functionalized surface group.

14. The polymeric nanoparticle of claim 13, wherein said functionalized surface group is an amine group, a thiol group, an alcohol group or a carboxylic acid group.

15. The polymeric nanoparticle of claim 13, wherein said functionalized surface group is bound to targeting ligand.

16. The polymeric nanoparticle of claim 15, wherein said targeting ligand is an antibody or a peptide.

17. The polymeric nanoparticle of claim 9, wherein said nanoparticle encapsulates one or more water-soluble agents.

18. The polymeric nanoparticle of claim 17, wherein said one or more water-soluble agents is selected from the group consisting of a small organic molecule drug, a DNA molecule, an RNA molecule, a protein, a fluorescent dye, a radioisotope, a contrast agent, and an imaging agent.

19. The polymeric nanoparticle of claim 9, wherein said nanoparticle encapsulates one or more water-insoluble agents.

20. The polymeric nanoparticle of claim 9, wherein said nanoparticle is less than about 200 nm in diameter.

21. The polymeric nanoparticle of claim 9, wherein said nanoparticle encapsulates paclitaxel.

22. The polymeric nanoparticle of claim 9, wherein said nanoparticle encapsulates gemcitabine.

23. The polymeric nanoparticle of claim 9, wherein said nanoparticle encapsulates a gadolinium complex or a gadolinium chelate.

24. The polymeric nanoparticle of claim 9, wherein said nanoparticle encapsulates iron oxide.

25. A polymeric nanoparticle produced by the process of claim 1, wherein sorbitol and itaconate are condensed to form said polymeric monomers and said cross-linker is ethylene glycol diitaconate.

26. A polymeric nanoparticle produced by the process of claim 1, wherein gluconic acid and acrylamidoglycolic acid are condensed to form said polymeric monomers and said cross-linker is glycerol dimethacrylate.

27. A process for producing a polymeric nanoparticle comprising:
(a) condensing one or more primary hydroxyacid compounds to generate a polyester;
(b) adding one or more cross-linkers selected from the group consisting of ethylene glycol diitaconate, glycerol (bis) itaconate, sorbitol diitaconate, glycerol dimethacrylate and divinyl citrate;
(c) initiating polymerization to generate a solid particle; and
(d) removing the solid particle from solution.

28. The process of claim 27, wherein said condensing occurs via esterification.

29. The process of claim 27, wherein said condensing occurs via enzyme catalysis.

30. The process of claim 29, wherein the enzyme is a lipase.

31. The process of claim 27, wherein said initiating in (c) occurs in the presence of one or more surfactants.

32. The process of claim 27, further comprising passing the removed solid particle through one or more porous filters to generate a nanoparticle that is less than about 200 nm in diameter.

33. The process of claim 27, further comprising adding an agent to be encapsulated to the solution prior to said initiation (c).

34. The process of claim 27, further comprising adding in (a) a functionalized monomer, thereby generating a functionalized group on the surface of the nanoparticle.

35. A polymeric nanoparticle produced by the process of claim 27.

36. The polymeric nanoparticle of claim 35, wherein said nanoparticle is biodegradable.

37. The polymeric nanoparticle of claim 35, wherein said primary hydroxyacid compound is selected from the group consisting of gluconic acid, hydroxy aliphatic acid, lactic acid, glycolic acid, acrylamido glycolic acid, hydroxy aromatic acid, salicylic acid, glyceric acid, threonic acid and glutathione.

38. The polymeric nanoparticle of claim 35, further comprising a functionalized surface group.

39. The polymeric nanoparticle of claim 38, wherein said functionalized surface group is an amine group, a thiol group, an alcohol group or a carboxylic acid group.

40. The polymeric nanoparticle of claim 38, wherein said functionalized surface group is bound to targeting ligand.

41. The polymeric nanoparticle of claim 40, wherein said targeting ligand is an antibody or a peptide.

42. The polymeric nanoparticle of claim 35, wherein said nanoparticle encapsulates one or more water-soluble agents.

43. The polymeric nanoparticle of claim 42, wherein said one or more water-soluble agents is selected from the group consisting of a small organic molecule drug, a DNA molecule, an RNA molecule, a protein, a fluorescent dye, a radioisotope, a contrast agent, and an imaging agent.

44. The polymeric nanoparticle of claim 35, wherein said nanoparticle encapsulates one or more water-insoluble agents.

45. The polymeric nanoparticle of claim 35, wherein said nanoparticle is less than 200 nm in diameter.

46. The polymeric nanoparticle of claim 35, wherein said nanoparticle encapsulates paclitaxel.

47. The polymeric nanoparticle of claim 35, wherein said nanoparticle encapsulates gemcitabine.

48. The polymeric nanoparticle of claim 35, wherein said nanoparticle encapsulates a gadolinium complex or a gadolinium chelate.

49. The polymeric nanoparticle of claim 35, wherein said nanoparticle encapsulates iron oxide.

50. A polymeric nanoparticle produced by the process of claim 27, wherein sorbitol and glycerol are condensed to form said polyester and said cross-linker is glycerol dimethacrylate.

51. A process for producing a polymeric nanoparticle comprising:
(a) condensing one or more primary dihydroxy compounds and one or more diacids to generate a polyester;
(b) adding one or more water-soluble cross-linkers;
(c) initiating polymerization to generate a solid particle; and
(d) removing the solid particle from solution.

52. The process of claim 51, wherein said condensing occurs via esterification.

53. The process of claim 51, wherein said condensing occurs via enzyme catalysis.

54. The process of claim 53, wherein the enzyme is a lipase.

55. The process of claim 51, wherein said initiating in (c) occurs in the presence of one or more surfactants.

56. The process of claim 51, wherein said water-soluble cross-linker is selected from the group consisting of lysine-diacrylamide, diethylenetriamine-diacrylamide, arginine-diacrylamide and 2,2′-oxydiethanol-diacrylate.

57. The process of claim 51, further comprising passing the removed solid particle through one or more porous filters to generate a nanoparticle that is less than about 200 nm in diameter.

58. The process of claim 51, further comprising adding an agent to be encapsulated to the solution prior to said initiation (c).

59. The process of claim 51, further comprising adding in (a) a functionalized monomer, thereby generating a functionalized group on the surface of the nanoparticle.

60. A polymeric nanoparticle produced by the process of claim 51.

61. The polymeric nanoparticle of claim 60, wherein said nanoparticle is biodegradable.

62. The polymeric nanoparticle of claim 60, wherein said primary dihydroxy compound is selected from the group consisting of sorbitol, mannitol, iditol, sucrose, fructose, lactose, ribose, maltose, glycerol, ethylene glycol, propylene glycol and glycerol.

63. The polymeric nanoparticle of claim 60, wherein said diacid is selected from the group consisting of itaconic acid, adipic acid, succinic acid, fumaric acid, and acylamidoglutamic acid.

64. The polymeric nanoparticle of claim 60, further comprising a functionalized surface group.

65. The polymeric nanoparticle of claim 64, wherein said functionalized surface group is an amine group, a thiol group, an alcohol group or a carboxylic acid group.

66. The polymeric nanoparticle of claim 64, wherein said functionalized surface group is bound to targeting ligand.

67. The polymeric nanoparticle of claim 66, wherein said targeting ligand is an antibody or a peptide.

68. The polymeric nanoparticle of claim 60, wherein said nanoparticle encapsulates one or more water-soluble agents.

69. The polymeric nanoparticle of claim 68, wherein said one or more water-soluble agents is selected from the group consisting of a small organic molecule drug, a DNA molecule, an RNA molecule, a protein, a fluorescent dye, a radioisotope, a contrast agent, and an imaging agent.

70. The polymeric nanoparticle of claim 60, wherein said nanoparticle encapsulates one or more water-insoluble agents.

71. The polymeric nanoparticle of claim 60, wherein said nanoparticle is less than about 200 nm in diameter.

72. The polymeric nanoparticle of claim 60, wherein said nanoparticle encapsulates paclitaxel.

73. The polymeric nanoparticle of claim 60, wherein said nanoparticle encapsulates gemcitabine.

74. The polymeric nanoparticle of claim 60, wherein said nanoparticle encapsulates a gadolinium complex or a gadolinium chelate.

75. The polymeric nanoparticle of claim 60, wherein said nanoparticle encapsulates iron oxide.

76. A process for producing a polymeric nanoparticle comprising:
(a) condensing one or more primary hydroxyacid compounds to generate a polyester;
(b) adding one or more water-soluble cross-linkers;
(c) initiating polymerization to generate a solid particle; and
(d) removing the solid particle from solution.

77. The process of claim 76, wherein said condensing occurs via esterification.

78. The process of claim 76, wherein said condensing occurs via enzyme catalysis.

79. The process of claim 78, wherein the enzyme is a lipase.

80. The process of claim 76, wherein said initiating in (c) occurs in the presence of one or more surfactants.

81. The process of claim 76, wherein said water-soluble cross-linker is selected from the group consisting of lysine-diacrylamide, diethylenetriamine-diacrylamide, arginine-diacrylamide and 2,2′-oxydiethanol-diacrylate.

82. The process of claim 76, further comprising passing the removed solid particle through one or more porous filters to generate a nanoparticle that is less than 200 nm in diameter.

83. The process of claim 76, further comprising adding an agent to be encapsulated to the solution prior to said initiation (c).

84. The process of claim 76, further comprising adding in (a) a functionalized monomer, thereby generating a functionalized group on the surface of the nanoparticle.

85. A polymeric nanoparticle produced by the process of claim 76.

86. The polymeric nanoparticle of claim 85, wherein said nanoparticle is biodegradable.

87. The polymeric nanoparticle of claim 85, wherein said primary hydroxyacid compound is selected from the group consisting of gluconic acid, hydroxy aliphatic acid, lactic acid, glycolic acid, acrylamido glycolic acid, hydroxy aromatic acid, salicylic acid, glyceric acid, threonic acid and glutathione.

88. The polymeric nanoparticle of claim 85, further comprising a functionalized surface group.

89. The polymeric nanoparticle of claim 88, wherein said functionalized surface group is an amine group, a thiol group, an alcohol group or a carboxylic acid group.

90. The polymeric nanoparticle of claim 89, wherein said functionalized surface group is bound to targeting ligand.

91. The polymeric nanoparticle of claim 90, wherein said targeting ligand is an antibody or a peptide.

92. The polymeric nanoparticle of claim 85, wherein said nanoparticle encapsulates one or more water-soluble agents.

93. The polymeric nanoparticle of claim 92, wherein said one or more water-soluble agents is selected from the group consisting of a small organic molecule drug, a DNA molecule, an RNA molecule, a protein, a fluorescent dye, a radioisotope, a contrast agent, and an imaging agent.

94. The polymeric nanoparticle of claim 85, wherein said nanoparticle encapsulates one or more water-insoluble agents.

95. The polymeric nanoparticle of claim 85, wherein said nanoparticle is less than about 200 nm in diameter.

96. The polymeric nanoparticle of claim 85, wherein said nanoparticle encapsulates paclitaxel.

97. The polymeric nanoparticle of claim 85, wherein said nanoparticle encapsulates gemcitabine.

98. The polymeric nanoparticle of claim 85, wherein said nanoparticle encapsulates a gadolinium complex or a gadolinium chelate.

99. The polymeric nanoparticle of claim 85, wherein said nanoparticle encapsulates iron oxide.

100. A method of treating a tumor in a mammalian patient comprising: administering to the patient a polymeric nanoparticle according to any one of claims 9, 25, 26, 35, 50, 60 and 85, wherein the polymeric nanoparticle encapsulates one or more cancer chemotherapeutic agents.

101. The method of claim 100, wherein the cancer chemotherapeutic agent is selected from the group consisting of gemcitabine and paclitaxel.

102. The method of claim 100, wherein the nanoparticle further encapsulates an imaging agent.

103. The method of claim 102, wherein the imaging agent is iron oxide.

104. The method of claim 102, further comprising imaging the polymeric nanoparticle in the patient.

105. A method of treating a tumor in a mammalian patient comprising:
(a) administering to the patient the polymeric nanoparticle of any one of claims 9, 25, 26, 35, 50, 60 and 85; and
(b) administering ionizing radiation to the patient,
wherein the polymeric nanoparticle encapsulates one or more radiation-sensitizing agents.

106. The method of claim 105, wherein the radiation-sensitizing agent is selected from the group consisting of gemcitabine, paclitaxel and carboplatin.

107. The method of claim 105, wherein the nanoparticle further encapsulates an imaging agent.

108. The method of claim 107, wherein the imaging agent is iron oxide.

109. The method of claim 107, further comprising imaging the polymeric nanoparticle in the patient.

110. A method of imaging a polymeric nanoparticle in a mammalian patient comprising:
(a) administering to the patient the polymeric nanoparticle of any one of claims 9, 25, 26, 35, 50, 60 and 85; and
(b) imaging the nanoparticle,
wherein the polymeric nanoparticle encapsulates one or more imaging agents.

111. The method of claim 110, wherein the imaging agent is iron oxide.

112. A pharmaceutical composition comprising one or more of the nanoparticles of any one of claims 9, 25, 26, 35, 50, 60 and 85, and one or more pharmaceutically acceptable carriers or excipients.
